(19) United States
(12) Patent Application Publication
(10) Pub. No.: US 2022/0389364 A1
(43) Pub. Date: Dec. 8, 2022

(54) NANOSTRAW WELL INSERT DEVICES FOR IMPROVED CELL TRANSFECTION AND VIABILITY

(71) Applicants: NAVAN TECHNOLOGIES, INC., Palo Alto, CA (US); THE BOARD OF TRUSTEES OF THE LELAND STANFORD JUNIOR UNIVERSITY, Stanford, CA (US)

(72) Inventors: Ryan T. SWOBODA, Orange Park, FL (US); Whitney BURKS, Stanford, CA (US); Sergio LEAL-ORTIZ, Hayward, CA (US); Amanda JONSSON, Stanford, CA (US); Nicholas A. MELOSH, Menlo Park, CA (US)

(73) Assignees: NAVAN TECHNOLOGIES, INC., Palo Alto, CA (US); THE BOARD OF TRUSTEES OF THE LELAND STANFORD JUNIOR UNIVERSITY, Stanford, CA (US)

(21) Appl. No.: 17/762,152

(22) PCT Filed: Sep. 21, 2020
(86) PCT No.: PCT/US20/51883

§ 371 Date: Mar. 21, 2022

Related U.S. Application Data
(60) Provisional application No. 62/903,653, filed on Sep. 20, 2019.

(51) Int. Cl. C12M 1/42; C12M 1/12; C12N 15/87

(52) U.S. Cl. CPC C12M 35/02; C12M 25/04; C12N 15/87; B82Y 5/00

(57) ABSTRACT

Described herein are nano straw well insert apparatuses (e.g., devices and systems) that include nanotubes extending through and out of a membrane so that a material can pass through the membrane from a fluid reservoir depot and into a cell grown onto the nanotubes when electrical energy (e.g., electroporation energy) is applied. In particular, the device, systems and methods described herein may be adapted for cell growth viability and transfection efficiency (e.g., >70%). These apparatuses may be readily integratable into cell culturing processes for improved transfection efficiency, intracellular transport, and cell viability.

### CROSS REFERENCE TO RELATED APPLICATIONS

This application claims priority to U.S. Provisional Patent Application No. 62/903,653, titled “NANOSTRAW WELL INSERT DEVICES FOR IMPROVED CELL TRANSFECTION AND VIABILITY,” filed on Sep. 20, 2019.

### STATEMENT AS TO FEDERALLY SPONSORED RESEARCH

This work was made with Government support under NSF award 1759075 (STTR II). The Government has certain rights in the invention.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD

This disclosure is related generally to transfection and the delivery of molecular species across a membrane. More specifically, this disclosure relates to apparatuses (e.g., devices and systems) for transfecting a molecular species across a cell membrane into a cell.

### BACKGROUND

Transfection, or generally the delivery of molecular species across the lipid bilayer and into the cytosol or nucleus, is a powerful analytical tool with many applications spanning cell reprogramming, intracellular imaging and sensing, molecular farming, siRNA knockouts, drug screening, and pharmaceutical therapies. To meet such a wide spectrum of application, biologists have developed conventional transfection approaches which broadly fall into biological, chemical, and physical classes, with physically mediated techniques dominating emerging technologies. These methods all revolve around traversing the membrane with as little detriment to the cell and as high efficiency as possible. However, despite almost half a century of technique innovation, development, and optimization, the transfection field lacks a universal tool for delivery into the cell; this is to say, existing methods all have advantages and disadvantages which depend highly on experimental designs and objectives. In principle, the ideal transfection technique would permit the efficient transport of cargo, irrespective of size or structure, into any cell type with high throughput and preservation of cell physiology.

VanDersarl et al. (U.S. Pat. No. 9,266,725) described a simple, yet elegant biomimetic innovation in the bionanotechnology field in 2012 involving the processing of commonly used nanoporous membrane filters into nanofluidic substrates containing cell-penetrating architectures, or nanostraws. Nanostraws are essentially metal oxide nanotube structures, with a diameter on the order of a 100 nm, embedded in polymer films. Nanostraw technology uniquely establishes non-destructive intracellular access in real-time, vital for the delivery, or extraction, of bioactive molecular cargo. Cells cultured on Nanostraw devices are spontaneously penetrated, providing a stable, external handle on the delivery of fluidic material species into, or out of, cells. Moreover, enhanced penetration in combination with electroporation has been suggested, which can in turn result in much higher delivery rates.

The work herein is motivated, from a much broader perspective, with the goal of translating nanostraw technology from primary users, i.e., researchers in an academic nanoscience lab, to secondary users such as adjacent academic biology labs that may accelerate both the development and real impact of the technology. The barrier to this sort of technology transfer in the field of nanobiotechnology is typically steep, as it requires expertise and collaboration in both nanotech and biotech. To this end, the methods and apparatuses described herein may provide repeatable and uniform device performances, measured in terms of transfection efficiency and cell viability. Further, the method and apparatuses described herein may provide nanostraw device fabrication and optimized performance across multiple cell types.

### SUMMARY OF THE DISCLOSURE

Described herein are apparatuses (e.g., devices and systems) that are configured to be used with or include nanotubes extending through and out of a membrane so that a material can pass through the membrane from a fluid reservoir depot and into a cell grown onto the nanotubes when electrical energy (e.g., electro-delivery energy) is applied. In particular, the apparatuses (device and systems) and methods described herein may be adapted for long term cell growth viability (>5 days) and transfection efficiency (e.g., >70%). These apparatuses may be readily integratable into cell culturing processes for improved transfection efficiency, intracellular transport, and cell viability. Other examples of nanostraw devices that may be used with the methods and apparatuses described herein in part and in combination may be found in PCT/US2017/036806, published as WO 2017/214541 which is herein incorporated by reference in its entirety.

For example, described herein are adapter apparatuses, e.g., devices and systems, configured for use with nanostraw well insert devices. In some variations the nanostraw well insert device may be included as part of the apparatus. These apparatuses, and methods of using them, may be configured as cell culture systems for long-term cell growth and transfection. For example, a system may include: an adapter configured to hold a nanostraw well insert device, the nanostraw well insert device comprising a membrane from which a plurality of nanostraws project by at least 0.1 microns, the adapter comprising: a chamber having a base, a band electrode on in the base, wherein the chamber is configured to securely hold the nanostraw well insert device so that the membrane is vertically offset from the base so that the plurality of nanostraws are in fluid communication with a reservoir depot between the base and the membrane, further so that the band electrode is laterally offset from the plurality of nanostraws, a cover comprising a top electrode; a first electrical contact on or in the adapter in electrical communication with the band electrode; and a second electrical contact on or in the adapter in electrical communication with the top electrode.

Surprisingly, the use of a band electrode results in significant and unexpected improvements in the efficiency of the electro-delivery of material into the cell and/or the survival of the cells.

The band electrode typically has a flat shape with an outer perimeter forming the band, and an open interior. For example, the band electrode may have ring-shape, a rectangular shape, a square shape a triangular shape, etc. In some variations the band electrode is substantially round (e.g., ring-shaped, oval-shaped, etc.). In some variations the membrane (or the region of the membrane including the nanostraws) may be positioned within (but offset from) the opening formed through the band electrode. For example, the band electrode may comprise a ring electrode. The adapter may be configured so that the band electrode does not underlie the plurality of nanostraws when the nanostraw well insert device is held within the adapter. For example, the chamber of the adapter may be configured to securely hold the nanostraw well insert device so that the inner diameter of the band electrode is larger than an outer diameter of the membrane. For example, an outside diameter of the band electrode may be smaller than an inside diameter of the base. An inside diameter of the band electrode may be 3 mm or more (e.g., 6 mm or more, 9 mm or more, etc.). The band electrode may be laterally offset from a perimeter of the membrane by at least 2 mm when the nanostraw well insert device is held within the adapter.

As mentioned, the system may include a nanostraw well insert device (e.g., an inset device having a membrane from which a plurality of nanostraws project by at least 0.1 microns).

The top electrode may be stylus electrode, a slab electrode, etc. The top electrode may have a diameter of 2 mm or less (e.g., 1 mm or less, 0.5 mm or less, etc.) at its widest dimension horizontal dimension. The top electrode may have a surface area of less than about 4 mm² (e.g., less than about 2 mm², less than about 1 mm², etc.) at its widest dimension horizontal dimension. The top electrode may be separated from an inner surface of the adapter by at least 1 mm (e.g., at least 2 mm, 3 mm, 5 mm, etc.).

The base may be configured to vent air between the reservoir depot a bottom of the nanostraw well insert. The adapter may further comprise an inlet configured to regulate the pressure of the reservoir depot in the base. The cover may be configured to engage with the chamber so that the top electrode is separated from the band electrode by between 0.25 cm and 1.25 cm.

In some variations, the reservoir depot includes a concave surface. The chamber may comprise a cylindrical housing.

Also described herein are methods of using any of the apparatuses described herein. For example, a method of culturing and/or electro-delivery of material into cells (e.g., transfecting cells) may include: culturing one or more cells on a nanostraw well insert device, wherein the nanostraw well insert device comprises a membrane, wherein a plurality of nanostraws project through the membrane and into the well by greater than 0.1 microns; placing the nanostraw well insert device into a chamber of an adapter so that the membrane is vertically offset from a base of the chamber with the plurality of nanostraws in fluid communication with a reservoir depot at the base, wherein the reservoir depot is in electrical communication with a band electrode so that the band electrode is laterally offset from the membrane; placing a cover over the chamber, so that a top electrode on or in the cover is within the chamber and is separated from the base electrode with the nanostraw well insert therebetween; applying electrical energy between the band electrode and the top electrode to deliver a material from the reservoir depot, through the plurality of nanostraws and into the one or more cells; and removing the nanostraw well insert from the adapter and culturing the one or more cells.

Any of these methods may include culturing the cells on the nanocell well insert. Alternatively, and of the methods and apparatuses described herein may be used for efficient electro-delivery of material into cells that are acutely placed onto the nanostraws of the insert, even without culturing the cells for any significant length of time.

As described above, placing may include the nanostraw well insert device into the adapter such that the band electrode is laterally offset from the outer diameter of the membrane. For example, placing may comprise pacing the cover over the base so that the top electrode is separated from the base electrode by between 0.25 and 1.25 cm.

In some variations it may be beneficial to apply electrical energy as a constant or pulses of constant current. For example, applying the electrical energy may comprise applying pulses of current. Alternatively or additionally, applying the electrical energy may include applying a constant or pulsed voltage (e.g., a voltage of between about 20V and 110 V). IN some variations applying the electrical energy comprises applying a voltage of 15V or more (e.g., 20 V or more, 25 V or more, etc.). Applying the electrical energy may comprise applying a pulse width of between 10 and 500 microseconds at a pulse frequency of between 10 Hz and 1 KHz (e.g., between 10 Hz and 500 Hz, between 10 Hz and 200 Hz, between about 10 Hz and 120 Hz, between about 30 Hz and 500 Hz, between about 30 Hz and 110 Hz, etc.).

Any of these methods may include imaging the cell in the nanostraw well insert device before, during and/or after electro-delivery.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIG. 1A schematically illustrates an example of a nanostraw well insert. FIG. 1B shows an exploded view of this insert, with exemplary dimensions shown.

FIG. 1C is another example of a nanostraw well insert having a tapered well (“tapered nanostraw well insert”).

FIG. 1D is an exploded view of the nanostraw well insert of FIG. 1C, showing exemplary dimensions.

FIG. 2A shows a schematic illustration of a regular-sized well for culturing, testing, and manipulating a large batch of cells. FIG. 2B shows a schematic illustration of a petite well for culturing, testing, and manipulating a small batch of cells. FIG. 2C shows a schematic perspective view of a nanostraw membrane attached to the bottom of a well so as to make a culture well.

FIG. 3A show a perspective view of a stylus electrode (top) and a band electrode (shown here as a ring electrode) (base) in a delivery precursor holder (e.g., an electro-delivery adapter). FIG. 3B shows a perspective view of the stylus electrode (top) and the ring electrode (base) shown in FIG. 3A from a different angle. FIG. 3C shows a top view of the stylus electrode (top) and the ring electrode (bottom) shown in FIGS. 3A-3B.

FIG. 4A shows the dimensions of a regular-sized well for culturing, testing, and manipulating a large batch of cells. The regular-sized well in this example has an inner diameter of 6 mm. FIG. 4B shows the dimensions of a petite well for culturing, testing, and manipulating a small batch of cells. The petite well has an inner diameter (ID) of 3 mm. FIG. 4C shows a perspective view of a delivery precursor holder (e.g., an electroporation adapter) with an inner diameter of ˜12 mm and configured to receive a regular well or a petite well. The electro-delivery adapter has a band electrode (in this example a ring electrode) inside the inner wall by the reservoir depot.

FIG. 4D shows a partial cut away schematic of a regular well with a slab electrode on top and a band electrode (e.g., ring electrode) on the base. FIG. 4E shows a partial cut away schematic of a petite well with a stylus electrode on top and a ring electrode on the base for use in a petite well.

FIG. 5A shows another schematic illustration of a nanostraw well insert for culturing and testing cells. FIG. 5A illustrates a general nanostraw well insert device during transfection. In this example, the device includes a polycarbonate tube with a nanostraw membrane attached to the bottom so as to make a culture well. Cells are cultured directly in the device well with media within the well. For transfection via electroporation, the device is placed into contact with the cargo reservoir beneath the membrane, containing the molecular species to be delivered (shown here as small spheres in solution; e.g., DNA/RNA in water). Electric pulses are fired across two platinum electrodes to open pores in the cell membrane, enabling intracellular access. (In FIG. 5A, the cells and nanostraws are not drawn to scale). FIG. 5B shows nanostraw imaged via scanning electron microscopy; straw length typically ranges from 1 to 3 microns and the outer diameter is on the order of 100 nm. FIGS. 5C and 5D illustrate false-colored SEM images of cryo-fixed CHO cell on nanostraw membrane to illustrate the practical device principle. Nanostraws such as these have been demonstrated to spontaneously penetrate the cell membrane, and the applied electrical pulses may enhance delivery efficiency.

FIGS. 6A-6C illustrates a process flow schematic for formation of a nanostraw well insertion device. In FIG. 6A, PCTE membranes are mounted on a p-type (100) silicon carrier wafer. FIGS. 6B.1 to B.4 illustrate fabrication of a membrane including nanostraws. FIG. 6C.1 to C.3 illustrate the assembly of nanostraw well insert devices ready for biological testing in culture plate.

FIG. 7 is a process flow showing an overview of the production of a nanostraw insert.

FIGS. 8A-8D illustrate atomic layer deposition of an alumina layer on a substrate. The process generally includes alternating pulses of reactive gaseous precursors to generate layer-by-layer film growth. In FIG. 8A the Al₂O₃ film process is initiated by addition of the aluminum carrying precursor (TMA, or trimethyl aluminum). In FIG. 8B, TMA precursor reacts with a naturally adsorbed surface hydroxyl group on the substrate to bind aluminum to oxygen and release methane as a byproduct. In FIG. 8C, after all, or mostly all, surface oxygen sites have been reacted with TMA precursor molecules, H₂O vapor is pumped into the chamber. In FIG. 8D, water molecules react with the surface alumina atoms to form a “monolayer” of alumina, Al₂O₃. This process is repeated until the desired film thickness is achieved.

FIG. 9A shows a Savannah 100 apparatus (Cambridge NanoTech) that may be used to grow an Al₂O₃ film. FIG. 9B shows a user interface for operating the device shown in FIG. 9A. In FIG. 9C, the pressure profiles are used to monitor precursor pulse and chamber purge characteristics for quality control purposes. FIGS. 9D and 9E show five-point ellipsometry thickness uniformity test reports, showing 10 nm+/−1 nm thick alumina films on silicon via a standard ALD deposition protocol.

FIGS. 10A-10D illustrates one possible mechanism for atomic layer deposition of oxide material onto polymer substrate with increasing number of cycles. In FIG. 10A, diffusion of gaseous precursor is blocked after a critical cycle number and uneven diffusion is expressed as surface roughness. The thickness of alumina deposition on silicon as measured via ellipsometry is comparable to thicknesses estimated from SEM, as seen in FIG. 10E. Thickness observations from SEM may be skewed from a few possible artifact sources: amorphous material from polymer outgassing may deposit during the ALD vacuum conditions, silver/gold sputtering for SEM preparation adds thickness, and significant surface charging during imaging. However, open-pores can readily be imaged via SEM and aid in quality control.

FIG. 11 is an SEM showing silica nanostraws (100 nm pore size). While cell viability on silica architectures was quite excellent, standard biological protocols for transfection were less efficient than comparable alumina straws.

FIGS. 12A-12C show the fabrication and characterization of hafnia nanostraws. In FIG. 12A, hafnia nanostraws were successfully fabricated using a Savannah 200 (Cambridge Nanotech). FIGS. 12B and 12C show SEM images at different magnifications of hafnia nanostraws, showing clean, open pores that appear to have uniform diameters along the entirety of the straw length.

FIGS. 13A-13D illustrate one method of assembling at least a portion of a nanostraw well insert apparatus as described herein. FIG. 13A shows a tubular body (e.g. a polycarbonate tube) and an adhesive (shown as double-stick tape); FIG. 13B shows the tubular body with the adhesive on the bottom surface; in FIG. 13C, the nanostraws on the substrate are attached via the double-sided tape to the tubular body. The insert is shown inserted into a well in FIG. 13D.

FIG. 14 illustrates an example of a standard transfection protocol for expression of nucleic acid delivery into model cell lines. In this example, the method typically takes 24 to 72 hours from sterilization to image analysis, depending on whether or not translocation is required for expression. Transfection steps include sterilization, cell culture, electroporation, cell incubation, and optical microscopy.

FIG. 15 illustrates a longer method of cell culture and transfection (e.g., taking longer than 5 days), including culturing in the improved nanostraw well insert apparatuses described herein.

FIG. 16A shows an example of an apparatus including a nanostraw well insert and an electro-delivery adapter or carrier. FIG. 16B shows a well insert being inserted into the base of an adapter/carrier; FIG. 16C shows the closed adapter/carrier holding a nanostraw well insert onto which cells have been cultured. The adapter/carrier and enclosed nanostraw well insert may be held and manipulated while protecting the cultured cells, including placing into an electro-delivery apparatus for delivery of electrical current.

FIGS. 17A-17H show the electro-delivery results of cargo composed of 20 uM red oligo in 400 ul TE buffer into cells. In all electro-delivery experiments, cells were suspended inside the wells in regular media buffer (RPMI1640, 10% FBS, 1× Pen Strep). FIG. 17A-FIG.C and FIG. 17E-FIG. 17G show electro-delivery results using petite wells and a stylus electrode on top and a band electrode (in this example, shown as a ring electrode) at the base as described herein. FIG. 17D and FIG. 17H) show electro-delivery results using regular-sized wells and a slab electrode on top and a ring electrode at the base as described herein.

FIGS. 18A-18H show the electro-delivery results of cargo composed of 20 uM red oligo in 400 ul TE buffer into cells. In all electro-delivery experiments, cells were suspended inside the wells in regular media buffer (RPMI1640, 10% FBS, 1× Pen Strep). FIG. 18A-FIG. 18C and FIG. 18E-FIG. 18G show electro-delivery results using petite wells and a stylus electrode on top and a ring electrode at the base as described herein. FIG. 18D and FIG. 18H show electro-delivery results using regular wells and a slab electrode on top and a ring electrode at the base as described herein. All images were taken with identical exposure and magnification using Zeiss Axiovert 200M microscope.

FIGS. 19A-19F show the electro-delivery expression results of cargo composed of 10 ug of pMaxEGFP plasmid resuspend in 30 ul of buffer TE (FIG. 19A, D) using regular cell pac for control or 50 ug of pMaxEGFP plasmid resuspend in 400 ul of buffer TE (FIGS. 19B, 19C, 19E, and 19F) into cells using a ring electrode as described herein. In all electro-delivery experiments, cells were suspended inside the wells in regular media buffer (RPMI1640, 10% FBS, 1× Pen Strep).

FIGS. 20A and 20B illustrate one example of the efficiency and viability, respectively, of delivery of mRNA into Jurkat cells using the nanostraw well inserts and ring electrodes as described herein.

FIGS. 21A-21D are images showing transfection of cells (Hek 293 cells) using mRNA (FIGS. 21A and 21C) or plasmid (FIGS. 21B and 21D) as described herein, using tapered nanostraw well inserts and ring electrodes.

FIG. 22A shows a perspective view of four stylus electrodes (top) and 4 tapered wells lowered into the fluid reservoir depots created by an array of band electrodes cut from a metal plate (base) in a delivery precursor holder (e.g., an electro-delivery adapter).

FIG. 22B shows a perspective view of the four stylus electrodes (top) and the ring electrode (base) shown in FIG. 22A from a front perspective.

FIG. 22C schematically illustrates a partial cut-away view of a tapered well lowered into a fluid reservoir depot created by a band electrode (e.g., ring electrode) on the base.

FIG. 23A shows a perspective view of four rod electrodes (top) and a 4 wells lowered into a fluid reservoir depot created by an array of band electrodes cut from a metal plate (base) in a delivery precursor holder (e.g., an electro-delivery adapter).

FIG. 23B shows a perspective view of the four rod electrodes (top) and the ring electrode (base) shown in FIG. 23A from a different angle.

FIG. 23C shows another perspective view of the system of FIGS. 23A and 23B, shown from the front.

FIG. 23D is a schematic view showing a partial cut away view of a well lowered into the fluid reservoir depot created by the band electrode (e.g., ring electrode) on the base.

### DETAILED DESCRIPTION

Transfection, or the transport and integration of external material, typically nucleic acids, into the cytosol and/or nucleus of a living cell, is an essential technique for a wide set of modern biological, biomedical, and biotechnological methods, including cell reprogramming, intracellular imaging and sensing, molecular farming, siRNA knockouts, drug screening, and pharmaceutical therapies.

In general, the methods and apparatuses (devices, systems, etc.) described herein may provide for electro-delivery of material into a cell. Electro-delivery may include electroporation, electrophoresis, and/or electro-osmosis. In the description provided, the use of these methods and apparatuses should be understood to generally and generically include electro-delivery when referring individually to electroporation, electrophoresis, electro-osmosis or other techniques for delivering material across a cell membrane or assisting in delivering materials across cell membranes.

VanDersarl et. al. have previously reported a simple biomimetic nanostructure, or “nanostraw,” that establishes continuous fluidic access into the cell interior for the purpose of intracellular delivery, and extraction, of molecular cargo. Nanostraws are metal oxide nanotube structures, with diameter on the order of 100 nm, embedded in widely used polymer membrane filters. Cells cultured on nanostraw devices are spontaneously penetrated, providing a stable, external handle on the delivery of fluidic material species into cells. Nanostraws have been demonstrated to successfully transmit molecules from ions to 6000 base pair DNA structures over relatively short time scales. Moreover, existing literature highlights the compounded effects of integrated electro-delivery with nanostraw access for delivery of cargos of interest. The stability, versatility, and non-invasive nature of the nanostraw platform position the technology as a likely candidate for a universal solution for intracellular access with the purpose of understanding the underlying biology of the cell.

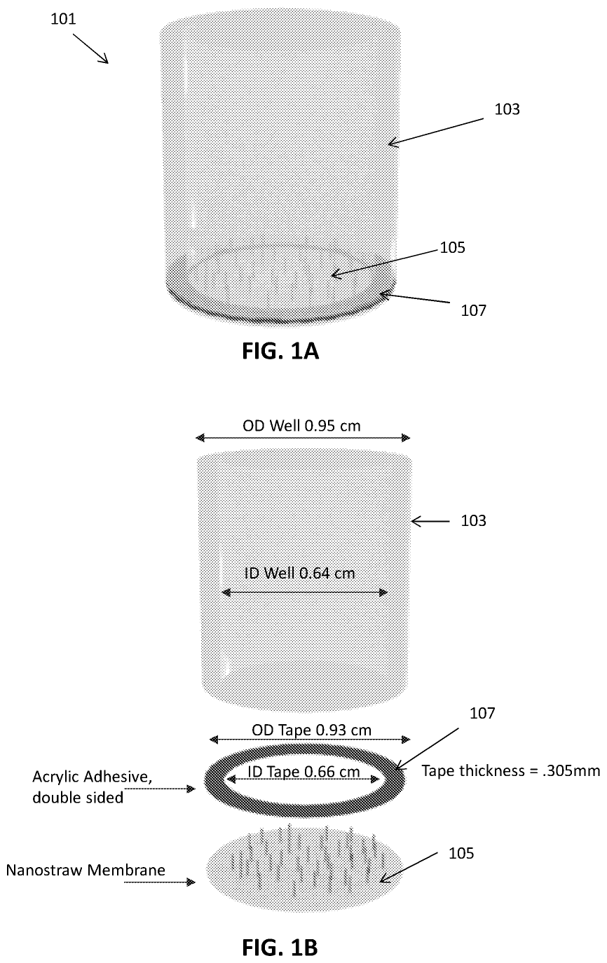

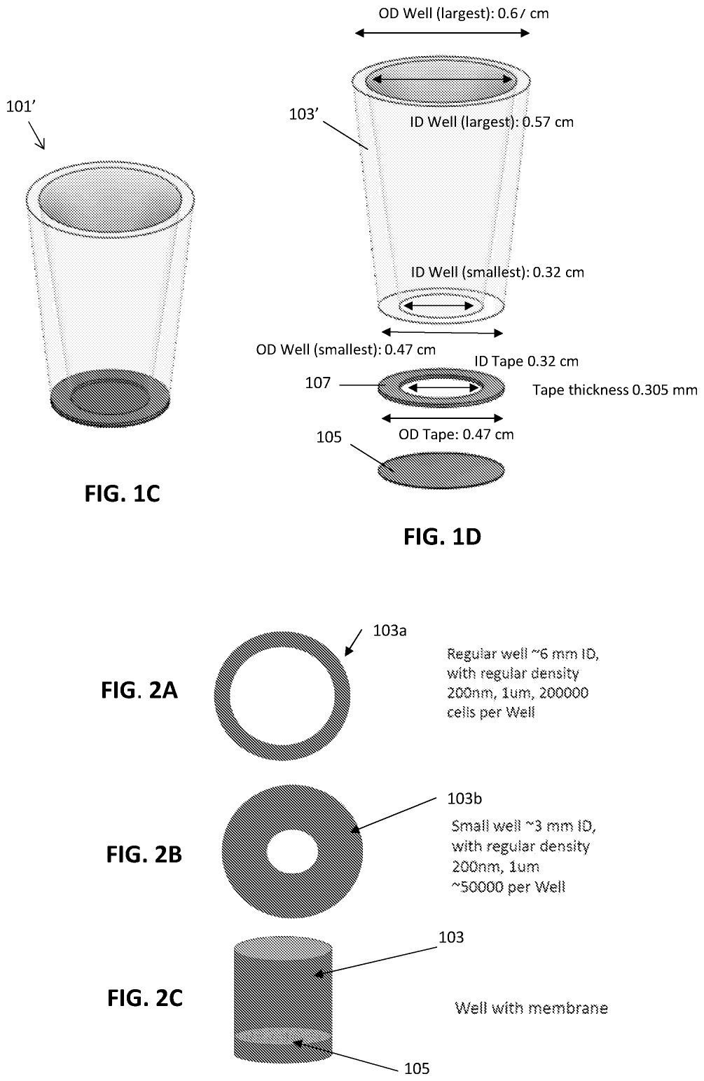

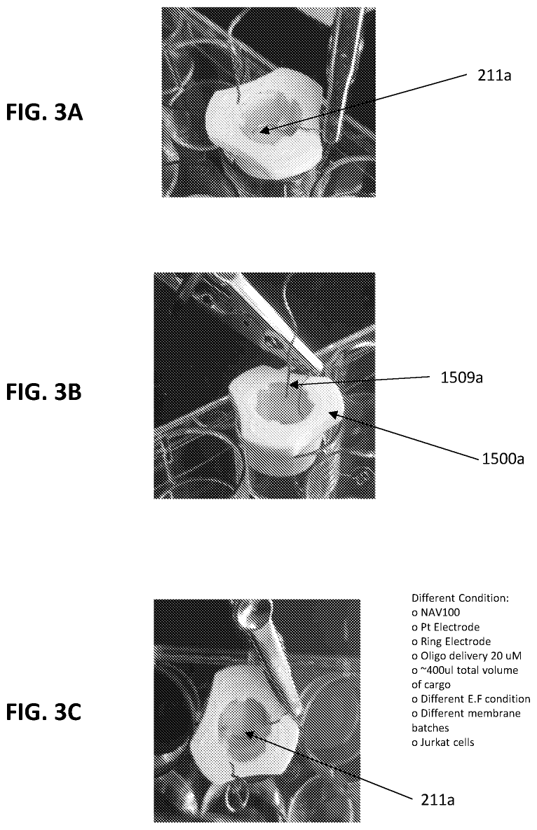

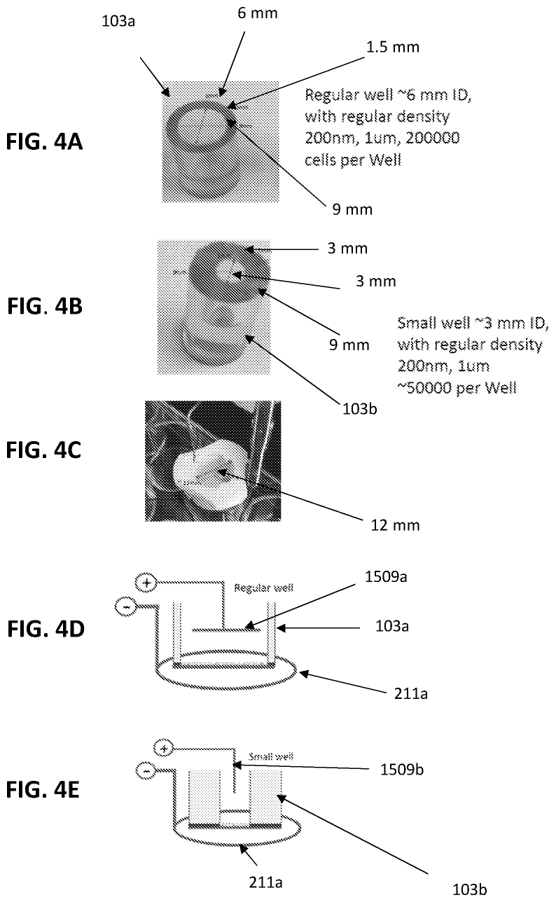

Described herein are nanostraw apparatuses (e.g. devices and methods) that may be particularly useful for electro-delivery cells with an external material such as nucleic acids, into the cytosol and/or nucleus of a living cell. In particular described herein are apparatuses including an electrode structure and electrode arrangement configured to effectively electroporate an external material into the cytosol and/or nucleus of a living cell. FIGS. 1A-1B, show a nanostraw well insert device 101. In this example, the nanostraw well insert device includes a cylindrical body 103 formed of an inert, preferably clear, material, such as polycarbonate. The nanostraw-containing substrate 105 is attached over the bottom of the cylinder by a double-sided tape 107, forming a well into which cells may be grown. The nanostraw material may, in particular, be nanostraws formed of a material such as hafnia. FIG. 1B is an exploded view of the nanostraw well insert device 101, showing exemplary dimensions for the cylindrical body 103, double-sided tape 107, and nanostraw membrane 105. FIGS. 2A-2B show variations of the cylindrical body 103 of the nanostraw well insert device. FIG. 2A shows a schematic top view of regular sized cylindrical body 103a. FIG. 2B shows a schematic top view of a petite cylindrical body 103b and FIG. 2C shows a cylindrical body with nanostraw membrane 105 in place. When in use, cells (not shown in this view) are contained within the walls of the cylindrical body 103. The regular sized cylindrical body 103a is configured to hold on the order of 200,000 cells per well. In some cases, it may be desirable to undertake a smaller sampling of cells for an experimental protocol or a smaller number of cells are available or a higher electroporation efficiency may be desired. For these situations, highly effective electrode configurations are described herein along with electroporation devices that may be especially useful with the electrode configurations. FIG. 2B shows petite cylindrical body 103b with a smaller inner diameter relative to the regular cylindrical body 103a. Petite cylindrical body 103b may be configured to hold on the order of 50,000 cells (e.g., between 200,000 and 25,000 cells, between 100,000 and 25,000 cells, between 50,000 and 25,000 cells, etc.) and still support effective electroporation. The wall of the cylindrical body 103 includes an outside surface and an inner surface with respective cylindrical body outer diameter and cylindrical body inner diameter. The outer diameter (OD; see FIG. 1B and see also FIG. 4A and FIG. 4B) of the wall of any well or cylindrical body 103 described herein (either regular or petite) may be configured to fit into a multi-well dish (e.g., a 2, 4, 6, 8, 16, 32, etc. multi-well dish). The outer diameter may be around 9 mm or around 10 mm. The inner diameter of the regular cylindrical body 103a may be, for example, around 6 mm while the inner diameter of the petite cylindrical body 103b may be around 3 mm (e.g., less than 6 mm such as between 5 mm and 1 mm, between 4 mm and 2 mm, etc.). The wall thickness of a cylindrical body may be from around 1 mm to around 4 mm or anything between these sizes (such as from 1 mm to 4 mm, from 2 mm to 4 mm, etc.). In some particular examples, the wall thickness of a regular cylindrical body may be around 1.5 mm and the wall thickness of a petite cylindrical body 103a may be around 3 mm. FIG. 3A-FIG. 3C show an electrode configuration effective for electroporation such as with a well insert device 101 with a cylindrical body 103. FIGS. 3A-3B (and see also FIG. 4C-FIG. 4E) show different perspective views of stylus electrode 1509b (top electrode) and ring electrode 211a (base electrode) in a holder 1500a (e.g., a delivery precursor or an electroporation adapter). FIG. 3C shows a top view of the stylus electrode 1509b and the ring electrode 211a (bottom) shown in FIGS. 3A-3B. The ring electrode 211a has an open center region. When the electrode 211 is ring shaped, its center is open or hollow and it may be a continuous, closed shape. The ring electrode 211a may run along the lower inner surface of the holder 1500a and may fit tightly against it or there may be a gap between the lower inner surface of the holder 1500a and the ring electrode 211a. The ring electrode 211a may have an outer diameter that is the same size as an inner diameter of the holder 1500a or may have a diameter that is smaller than an inner diameter of the holder 1500a. The cross section of the ring electrode 211a may be configured to fit within the holder 1500a and the ring electrode 211a (and a transverse cross section of the holder) may be round, oval, triangular, rectangular, square, pentagonal, octagonal, etc. though most commonly is round. The ring electrode 211a may be substantially any of these shapes such that small variations in the shape are allowed. Although a center chord across a circular ring electrode is referred to herein as “diameter” for simplicity, an axis or chord (e.g., a major or minor axis in an oval, etc.) in one of the other geometrically shaped electrodes described above may have a similar relationship to the holder 1500a as does the diameter. The ring electrode 211a has both an inner surface and an outer surface and has a corresponding inside diameter and a corresponding outside diameter. The inside diameter of the ring electrode 211a may be about or greater than 3 mm, about or greater than 6 mm, and/or about or greater than 9 mm, and may be about or less than 12 mm, about or less than 9 mm, about or less than 6 mm or anything in between these sizes (e.g., greater than 3 mm and about or less than 12 mm, greater than 6 mm and about or less than 12 mm, etc.). FIG. 4D and FIG. 4E shows ring electrode 211a may be larger around than the cylindrical body 103a or the cylindrical body 103a of the well insert device 101. The inside diameter of ring electrode 211a may be larger than a diameter of the inside wall of cylindrical body 103. The inside diameter of ring electrode 211a may be larger than a diameter of the outside wall of cylindrical body 103. The ring electrode is a base electrode 211 and may be made of platinum, may be negative, etc. as described elsewhere herein for any base electrode (e.g., base electrode 211). Any of the ring electrodes descried herein may alternatively be configured in some variations as disc electrodes that do not include an open middle region.

In general, the electrode on the base, such as the ring electrode or plate electrode (e.g., disc electrode) may be referred to as the band electrode. These electrodes may have any thickness, including varying thickness.

As described herein, ring electrodes may be particularly useful because the diameter of the ring region may be larger than the inner diameter (and/or outer diameter) of the base of the well insert, and any of these apparatuses may be configured so that the base (band) electrode is offset from the bottom of the well insert when the system is assembled, to prevent bubbles that may be formed when applying energy (e.g., applying a voltage) to the band electrode within the solution do not block the nanostraws in the membrane forming the bae of the well insert. Thus, the base of the well insert may be both vertically and horizontally offset from the band electrode (e.g., ring electrode).

FIGS. 1C and 1D illustrate another example of a nanostraw well insert device 101′, having a tapered well. In this example, the nanostraw well insert device includes a tapered cylindrical body 103′ formed of an inert, preferably clear, material, such as polycarbonate. In this example, the tapered body has an OD at the top (open) well of approximately 0.67 cm and an ID of approximately 0.57 cm; the ID of the well at the bottom of the taper (the bottom of the well 103′) is approximately 0.32 cm and the OD is approximately 0.47 cm. These dimensions are exemplary only. The nanostraw-containing substrate 105 is attached over the bottom of the cylinder by a double-sided tape 107, forming a well into which cells may be grown, as described above. FIG. 1D is an exploded view of the nanostraw well insert device 101′, showing exemplary dimensions for the cylindrical body 103′, double-sided tape 107, and nanostraw membrane 105.

FIG. 3A-FIG. 3C also show a top electrode in the form of stylus electrode 1509a. Similar to as indicated above for the ring electrode, the stylus electrode 1509a is a top electrode and may be made of platinum, may be positive, etc. as described elsewhere herein for any top electrode (e.g., electrode 211). The stylus electrode 1509a may have a relatively small end. The stylus electrode 1509 may have a relatively constant diameter. Although a transverse (e.g., horizontal) cross section of the stylus electrode 1509a may be round, oval, triangular, rectangular, square, pentagonal, octagonal, etc. most commonly it is round. The stylus electrode 1509a (e.g., at its end) may have a diameter that is less than 2 mm in its widest transverse dimension, may have a diameter that is less than 1 mm in its widest transverse (horizontal) dimension, or less than 0.5 mm in its widest transverse dimension. The stylus electrode 1509a (e.g., at its end) may have a cross-sectional surface area less than about 4 mm² at its widest transverse (horizontal) dimension, less than about 2 mm² at its transverse (horizontal), or less than about 1 mm² at its widest transverse (horizontal) dimension. The stylus electrode 1509a may be separated from an inner surface of the adapter by at least 1 mm, by at least 2 mm, or by at least 3 mm. FIG. 4D shows another electrode variation. FIG. 4D shows ring electrode 211a in conjunction with slab electrode 1509a (top electrode) and regular cylindrical body 103a. Slab electrode 1509a is a top electrode and may be platinum, may be attached over the insert, etc. as described herein for any other top electrodes (e.g., top electrode 1509).

The ring electrode described herein may be especially effective for electroporating cells. Although a base electrode 211 (as described elsewhere herein) may be a planar electrode that covers much of the bottom of the nanostraw well insert device and delivers transfection efficiencies greater than 70-90% under defined conditions, the ring electrode utilizes different parameters with similar or greater efficiencies. For example, the nanostraw cell culture system described herein may deliver efficient transfection under an applied voltage of around or more than 15 V, around or more than 20 V, around or more than 30 V, around or more than 40 V, around or more than 50 V. For example, the nanostraw cell culture system described herein may deliver efficient transfection with a frequency of around or more than 40 Hz, around or more than 50 Hz, around or more than 60 Hz, around or more than 70 Hz, around or more than 80 Hz, around or more than 90 Hz, or around or more than 100 Hz, less than 100 Hz, less than 90 Hz, less than 80 Hz, less than 70 Hz, less than 60 Hz, less than 50 Hz or anything between these amounts.

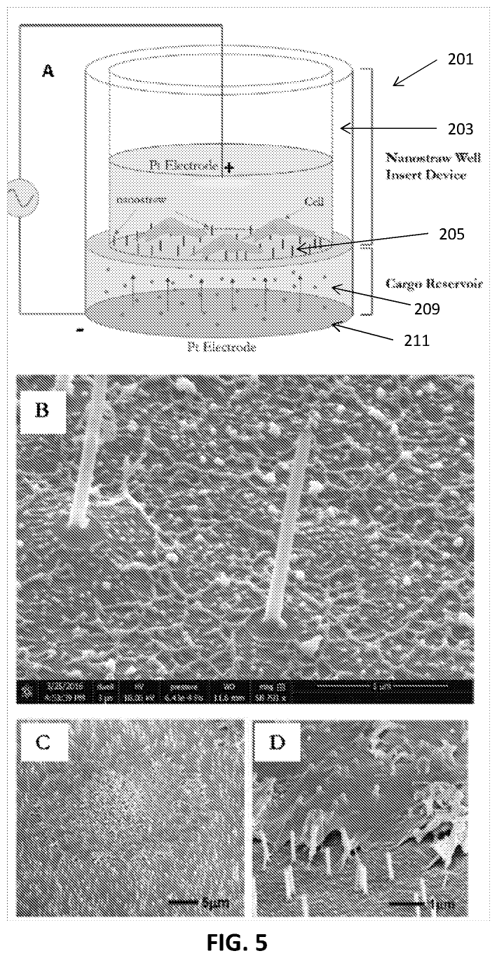

FIGS. 5A-5D illustrate another example of a nanostraw well insert apparatus. In FIG. 5A, a generic nanostraw well insert device is show during transfection. The insert device may be used with a matching delivery precursor holder (e.g., an electroporation adapter/cell cap), as mentioned. In FIG. 5A, the inset portion of the apparatus consists of a polycarbonate tube 203 with a nanostraw membrane 205 attached to the bottom so as to make a culture well, as described in reference to FIG. 1A-1B, above. Cells may be cultured directly in the device well with media (the upper media is “regular media,” as described above in the transfection protocol). The insert apparatus may be nitrated into a delivery precursor holder (e.g., an electroporation adapter/cell cap) or the holder/adapter may be separate. In FIG. 5A, for transfection via electroporation, the insert is placed into contact with a cargo reservoir 209 containing the molecular species to be delivered (shown here as small spheres in solution; e.g., typically, DNA/RNA in water). The cargo reservoir may be formed in the holder/adapter and may be configured to prevent bubbles forming between the base electrode 211 and the bottom of the membrane of the insert, as this may deleteriously affect transfection.

Electric pulses may be fired between the top electrode and the bas electrode, which are shown in this example as platinum electrodes; this may open pores in the cell membrane, enabling intracellular access through the nanostraws.

FIGS. 5A-5D show scanning electron microscopy (SEM) of nanostraws that may form part of the nanostraw membrane, including cells growing on them. In this example, the straw length typically ranges from 1 to 3 microns and the outer diameter is on the order of 100 nm. In FIG. 5C-5D, the SEM image shows a cryo-fixed CHO cell on nanostraw membrane to illustrate the practical device principle. Nanostraws have been demonstrated to spontaneously penetrate the cell membrane and electrical pulses act as “valves” to enhance delivery efficiency.

FIG. 2A shows a schematic illustration of a regular-sized well for culturing, testing, and manipulating a large batch of cells. FIG. 2B shows a schematic illustration of a petite well for culturing, testing, and manipulating a small batch of cells. FIG. 2C shows a schematic perspective view of a nanostraw membrane attached to the bottom of a well so as to make a culture well.

FIG. 3A show a perspective view of a stylus electrode (top) and a ring electrode (base) in a delivery precursor holder (e.g., an electroporation adapter). FIG. 3B shows a perspective view of the stylus electrode (top) and the ring electrode (base) shown in FIG. 3A from a different angle. FIG. 3C shows a top view of the stylus electrode (top) and the ring electrode (bottom) shown in FIGS. 3A-3B.

FIG. 4A shows the dimensions of a regular-sized well for culturing, testing, and manipulating a large batch of cells. The regular-sized well in this example has an inner diameter of 6 mm. FIG. 4B shows the dimensions of a petite well for culturing, testing, and manipulating a small batch of cells. The petite well has an inner diameter (ID) of 3 mm. FIG. 4C shows a perspective view of a delivery precursor holder (e.g., an electroporation adapter) with an inner diameter of ˜12 mm and configured to receive a regular well or a petite well. The electroporation adapter has a ring electrode inside the inner wall by the reservoir depot.

FIG. 4D shows a partial cut away schematic of a regular well with a slab electrode on top and a ring electrode on the base. FIG. 4E shows a partial cut away schematic of a petite well with a stylus electrode on top and a ring electrode on the base for use in a petite well.

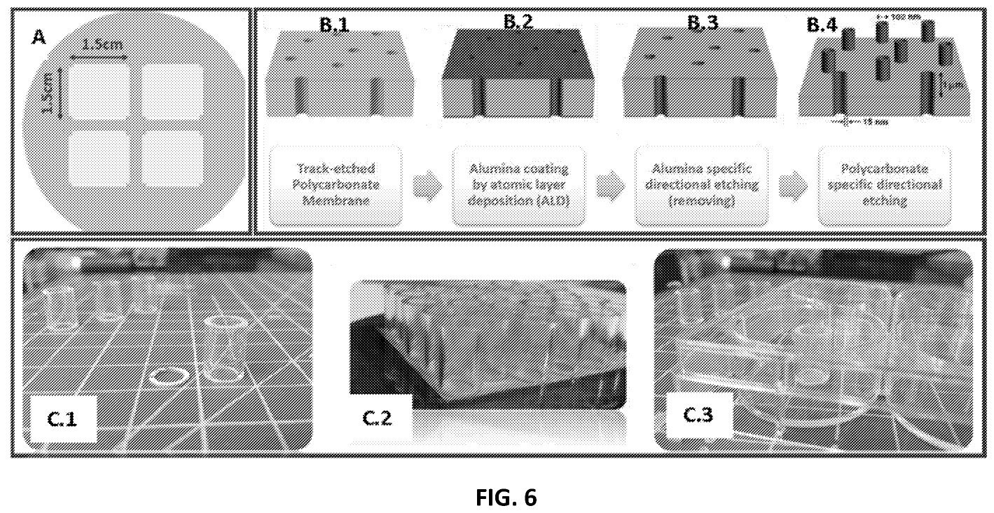

FIG. 6, B1-B4 illustrates an overview of nanostraw membrane fabrication, showing a three step process. Track-etched membrane filters (B.1) are coated via metal oxide atomic layer deposition (B.2). Following the deposition process, the top metal oxide surface layer is reactive ion etched in plasma (B.3). Nanostraw length is controlled via oxygen plasma etching (B.4), selective to the supportive polymer matrix. Provided the starting track-etched membrane pore sizes are selected, all Nanostraw dimensions can be tailored: straw length, density, outer and inner diameter.

Despite consistent confirmation of delivery/extraction of materials across the cell-membrane for model cells (e.g., CHO, HEK293), straw-cytosol spontaneous penetration events have been recently reported as occurring stochastically with a probability of less than 10% (˜5 to 15 penetrations/cell). While continuous, leaky access may be undesirable and detrimental to the cell, increased penetration requires an induced field which can certainly complicate design and optimization. In particular, electroporation has demonstrated to increase penetration and delivery events; however, internal observations have reported relatively non-uniform spatial transfection efficiencies (TE) with respect to centimeter-squared device areas (˜100,000 cells/monolayer). Relatively improbable spontaneous penetration and spatially non-uniform transfection efficiencies with respect to an induced field have proven to be problematic for existing device fabrication.

Described herein are nanostraw apparatuses and methods that may address many of these issues. For example, the methods and apparatuses described herein may be used with a wide variety of biological applications, such as immune cell reprogramming and stem cell modification. The nanostraw tools described herein may have enhanced uniformity and, in particular, may provide increased cell viability compared to prior devices.

The production of nanostraw well inserts for intercellular delivery is a five step process: (1) mounting and positioning of track-etched polymer membrane filters on silicon carrier wafers (FIG. 6A); (2) conformal atomic layer deposition (ALD) of metal oxide onto track-etched nanoporous polymer membrane (FIGS. 6 B.1 and B.2); directional ion etching of the top surface of ALD to expose polymer (FIG. 6 B.3); oxygen plasma etching of polymer membrane to control nanostraw length (e.g., 1 to 3 microns) (FIG. 6 B.4); and assembly of nanostraw well insert devices via adhesive annular tape and trimming of excess nanostraw membrane (FIGS. 6, C.1 to C.3).

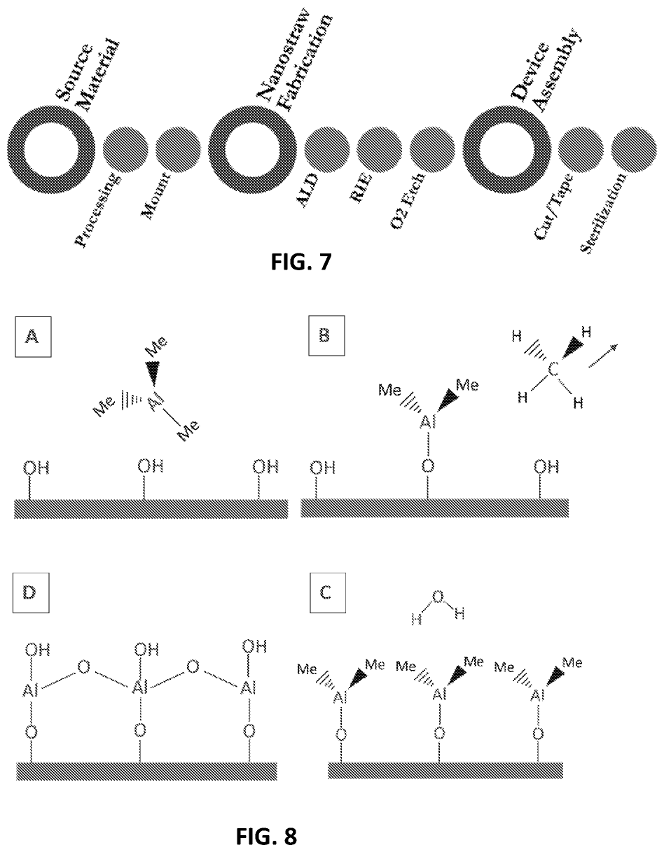

As illustrated in FIG. 7, the production of a nanostraw insert can be broken down into three production nodes based on considerations with respect to fixed dimensions, flexible dimensions, and form factor. Commercially available polymer membranes provide a range of fixed starting dimensions such as nanostraw density (e.g., 106 pores/cm2 to 109 pores/cm2), outer straw diameters (e.g., 50 nm to 10 μm), and membrane thicknesses (e.g., 5 μm to 20 μm). Flexible dimensions/properties such as wall thickness, inner diameter, straw height, roughness, tip sharpness, and biological compatibility (functionalization) are governed by the material choice for ALD as well as the respective deposition and etching processing parameters. See, e.g., FIGS. 5A-5D. While a variety of microfluidic designs may be employed, the nanostraw well insert apparatuses described herein may be configured as described herein.

FIG. 7 is a process flow showing nodes of a nanostraw well insert production. Starting with the source material (step I) preparation, then nanostraw fabrication (steps II, III, and IV), and concluding with device assembly (step V). The first node (i.e. source material) may include choice and preparation of polymer material and/or density. Processing steps at the second node (i.e. nanostraw fabrication) can be tailored to meet nanostraw dimensions such as inner diameter, length and straw material. The third node (device assembly) may set the device form factor.

Compared to prior methods, the methods described herein may yield both uniform nanostraw well devices and transfection efficiencies consistently over 75 percent with standard cell lines.

A track etched polymer may be chosen depending on relative availability and industrial experience. Ultimately, the polymer material must be compatible with cell culturing, deposition, and plasma process conditions, as discussed below. Polycarbonate (PC) track etched (PCTE) membranes have been used for large scale filtration and cell culture applications. Hence, PCTE is one example of a nanoporous membrane substrate for nanostraw fabrication described herein. In addition to PCTE also described herein are substrates of PET, or polyethylene terephthalate, which the inventors have identified as suitable for fabrication, culturing, and imaging conditions due to its amenable glass transition temperature and high degree of transparency.

In one example, the roll-to-roll manufacturing of PC films is an exemplary process that may be used. The process consists of extruding PC films to a prescribed thickness (˜5-20 μm), exposing the film to beta particle penetration of a particular density (2-4×107 pores/cm2 for nanostraw fabrication). Once pores have been introduced, process engineers wet etch the pores in a combination of UV light and basic solution (1M NaOH) to a desired diameter (<100 nm), and modifying with selected wetting agents to tune hydrophobicity. An example of a typically used wetting agent is polyvinylpyrrolidone (PVP) which is used to increase surface hydrophilicity for cell culture.

Upon retrieval of processed PCTE and prior to additional processing steps, membranes may be cut into 1.5 cm×1.5 cm square pieces using surgical scissors, positioned in quadrants on p-type (100) silicon wafers and mounted at the corners via Kapton processing tape. The square length is determined by the size of the nanostraw well device such that four devices can be produced per membrane square, i.e. 16 devices/wafer. Despite sacrificing some interstitial membrane material to device fabrication, membranes are positioned as such for logging within-batch processing conditions to further develop quality assurance protocols. Moreover, the leftover interstitial membrane material may be used for destructive characterization such as SEM. Silver sputtering has been observed to change the nanostraw surface chemistry which effectively renders nanostraws cytotoxic. However, in the direction of developing quality control procedures, gold (Au) sputtering may potentially be used examine the link between straw morphology and transfection performance as gold has been demonstrated to be more biocompatible.

Atomic layer deposition (ALD) is a chemical vapor deposition technique that can be enhanced thermally and/or by the utilization of plasma (PEALD) or radical species. The atomic layer epitaxy, or ALE, technique was modified in the late 1990's to include non-epitaxial deposition and has been recently referred to as atomic layer deposition. ALD is commonly used in a variety of thin film technologies, including but not limited to metal oxide high-k gate materials for MOSFET technologies and abrasive/protective coatings for polymers. The development and enabling of ALD over the recent years can be characterized by advancements in scalable processing techniques and a widening range of processing materials and precursors, both of which have expanded the market for ALD applications in terms of cost-effectiveness and material compatibility.

The fundamental principle for ALD is the layer-by-layer growth of films on a heated substrate. In this deposition method, two chemical precursors are selected for their high relative reactivity and introduced to the process chambers sequentially so as to control reaction at the surface. In principle, each reaction step is self-terminating. The first step involves exposing the substrate surface to the first reactant precursor and then pumping the reactant away. During this exposure the first reactant effectively leaves behind a “monolayer” of molecules adsorbed to the substrate surface. The chamber is then evacuated and a second reactant is introduced into the chamber. This second precursor reacts with the monolayer of the first reactant, forming one layer (typically less than a full layer) of the solid film being sought. After this, the remaining second reactant and any gas phase reaction products are removed from the chamber. This process, diagrammatically illustrated in FIG. 8A-8D, is repeated as many times as necessary to grow a film of the desired thickness.

While atomic layer deposition is traditionally marked by rather slow deposition rates (on the order of an angstrom/min) relative to other chemical vapor deposition techniques, the advantages of ALD include: “digital” thickness control on the atomic level (e.g., film thickness can be atomically controlled by varying the number of ALD cycles); relatively low temperatures and pressures, which enables compatibility with less robust substrates, such as polymers and some papers; conformal film growth over high aspect ratios on topographical substrates (gas precursors can reach any exposed surface, i.e. coverage is not limited by line-of-sight vapor source, in the case of non-PEALD processes); and relatively cheap deposition with an economy of scale. Ultrafast deposition (˜0.5 nm/sec, on the order of 300× the rate of traditional ALD) is may be achieved with roll-to-roll coating and parallel spatial processing.

The nanostraw production described herein may exploit this conformal film deposition, along with selective etchings, to leverage nanometer resolution for growth of high-aspect ratio structures.

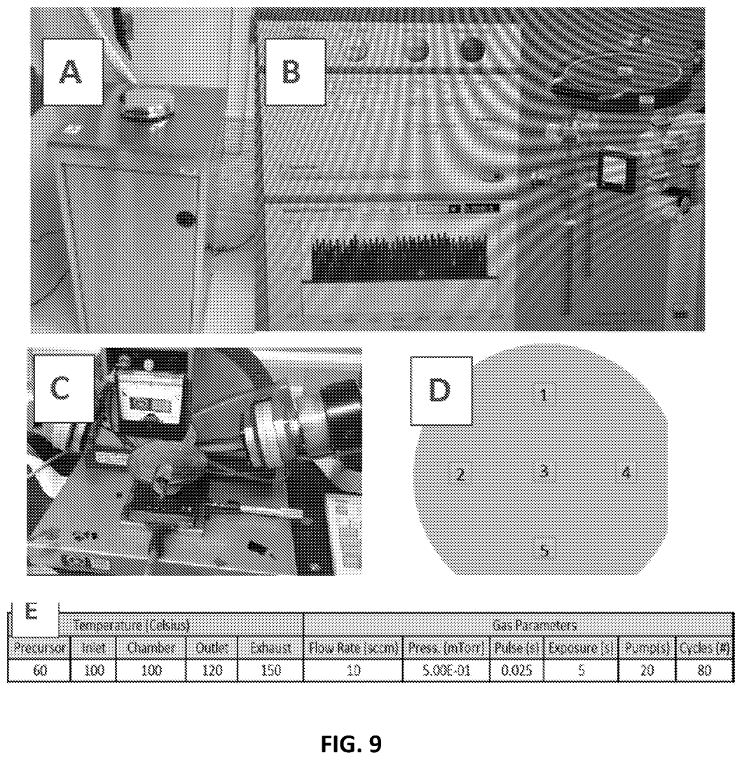

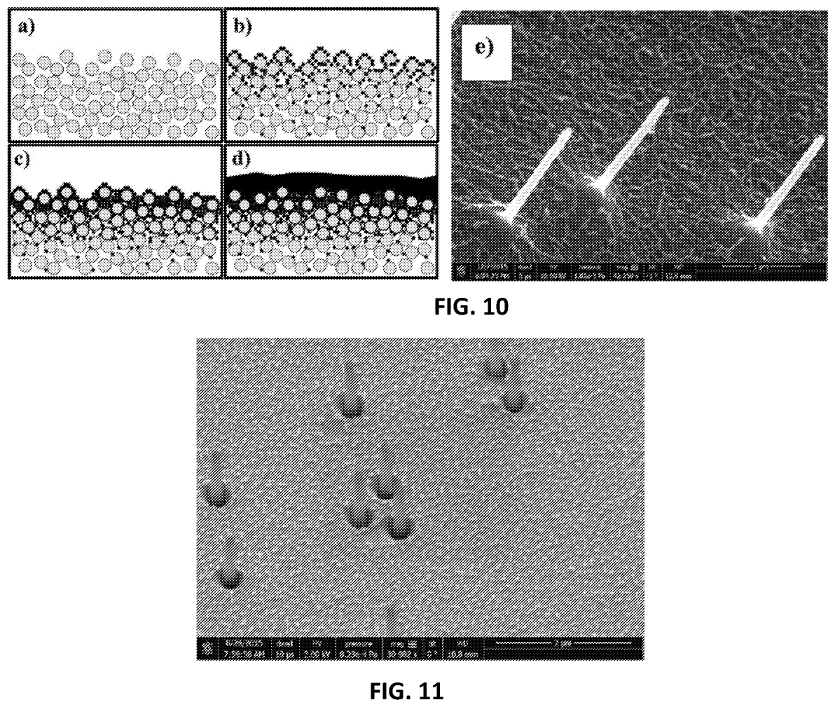

The parameters for controlling the ALD process may include temperature set points throughout the system, precursor (TMA and H2O for Al2O3) flow characteristics, and delay time for system equilibration. Film uniformity may be enhanced with oxygen plasma cleaning of the membranes prior to initiating the ALD process. The standard film growth parameter used yields an approximate deposition of 10 nm+/−1 nm with a wafer uniformity within 5% on p-type silicon (see, e.g. 5 point analysis in FIG. 9D). Al2O3 film thicknesses on silicon were measured via ellipsometry with three angstrom resolution. Alumina deposition thickness on silicon was used to correlate to deposition thickness on polycarbonate. Actual ALD thickness was measured as the side wall thickness of fully fabricated nanostraws using an FEI scanning electron microscope (see, e.g., FIG. 10E, normal to electron beam).

In FIG. 9, a standard Al2O3 film was grown using a Savannah 100 apparatus (Cambridge NanoTech), shown in FIG. 9A. FIG. 9B, shows a user interface for operating the device shown in FIG. 9A. In FIG. 9C, the pressure profiles are used to monitor precursor pulse and chamber purge characteristics for quality control purposes. FIGS. 9D and 9E show five-point ellipsometry thickness uniformity test reports, showing 10 nm+/−1 nm thick alumina films on silicon via a standard ALD deposition protocol.

When depositing thin films via atomic layer deposition (<10 nm), it is often important to consider the substrate surface properties. Particularly in the case of ALD on polymers, surface roughness can be attributed to the initial ALD cycles diffusing into the polymer material (15 to 30 cycles) before reaching self-limiting, layer-by-layer growth. Beyond a critical number of cycles, the ALD material eventually acts as a diffusive barrier into the polymer and sequential growth may proceed. The mechanism for ALD nucleation/growth on polymers, as indicated by literature, is illustrated in FIG. 10A-FIG. 10D.

From experimental observations, impedance spectroscopy, and SEM, the actual deposition thickness is believed to range somewhere between 15 to 30 nm, approximately corresponding with 10 nm of deposition on silicon wafer measured via ellipsometry (see, e.g., FIG. 10E). The discrepancy between these thickness estimates may be rationalized through consideration of the mechanism illustrated in FIGS. 10A-10E; the diffusive front of TMA precursors during the initial nucleation phase may effectively add to the desired 10 nm deposition to be expected if there were already a diffusive barrier on the polymer. SEM on high-aspect ratio, insulative materials often comes with artifacts in the form of sputter material thickness and surface charging from the electron beam. This is to say, SEM estimates of ALD thickness should be understood qualitatively.

Biological surface interactions occur predominantly on the outer wall of nanostraws. There is evidence to support a strong correlation between cell adhesion and surface roughness, and our models indicate surface roughness from the ALD process may be actually be beneficial for nanostraw-cell penetration. This is consistent with observations of spatially uniform device transfection efficiencies within batches.

As mentioned above, nanostraws have previously been described, and optimized, from alumina. Surprisingly, it has been found that nanostraws having desirable properties may be formed of other materials, including silica or hafnia (HfO2). These materials may be more easily scaled, and may also provide improved performance, both for cell viability in culture and also for transfection efficiency.

Silica (SiO2) nanostraws were anticipated to perform well in terms of both TE and CV due to silica's observed biocompatibility and surface chemistry. Surprisingly, although the biocompatibility of such straws was high, the transfection efficiency was remarkably low. The silica crystal structure can readily be functionalized to direct cell compatibility via silane chemistry. Further, the isoelectric point, or the pH at which the surface charge of the material in aqueous solution is neutral, is significantly lower than that of alumina (˜2-3 vs ˜8-9).

It may be expected that the isoelectric points of the nanostraw material may be related to the properties of the material when operating as a nanostraw because solution conditions for nanostraw-mediated delivery must be biocompatible, i.e. buffered at pH˜7, and should not capture (or prove “sticky”) for even charged materials being passed into the cells through the straws. The silica surface may be negative charged whereas alumina surface would be positive with respect to the bulk solution. In the case of alumina, a positively charged surface may have unfavorable implications for transport of negatively charged nucleic acids, i.e. plasmid DNA, as this cargo may have a high probability of binding, and clogging within the nanostraw during delivery protocols.

FIG. 11 illustrates an example of a silica nanostraw material. Silica nanostraw fabrication was shown for 100 nm pore size nanostraws. While cell viability on silica architectures was excellent, standard biological protocols for transfection proved less optimal. Silica ALD on polycarbonate also had a reduced compatibility between precursor gas and polymer substrate. The adsorption coverage time was slower for silica on polycarbonate than alumina on polycarbonate. This difference in adsorption time may be attributed to a higher activation barrier for silica precursor adsorbed to polycarbonate than aluminum precursor adsorbed to polycarbonate. To account for this adsorption barrier, while not breaching the melting point of polycarbonate (˜150 degrees C.), the silica atomic layer deposition was plasma enhanced to better suit the redox surface chemistry. The precursors TDMAS, or tris(dimethlyamino)silane, and H2O, were used in the plasma phase at the interface with the substrate for substantial adsorption coverage. Material species in the plasma phase behaved differently than in the gas phase. One challenge with this protocol was enabling the silicon-carrying plasma to reach down into the straws to ensure complete step coverage and conformal coating before proceeding to the next cycle step. This is typically combatted by higher plasma doses to the layer (˜104 L). SiO2 ALD on polycarbonate for nanostraw devices has been demonstrated in an ALD window of 60-100 degrees Celsius. However, the link between higher plasma doses and the atomic scale surface roughness of the silica layer has yet to be fully explored in the context of preserving polycarbonate membrane quality during processing.

Surprisingly, hafnia (HfO2) nanostraws were found to be advantageous, even as unexpectedly compared with Alumina. Low temperature hafnia (HfO2) ALD parameters for PCTE membranes are similar to alumina parameters. Hafnia precursors, tetrakis (diethlyamino) hafnium (TDEAH) and H2O, may be thermally enhanced as previously described for alumina nanostraws. For example, the Savannah 200 ALD system (see FIG. 9A, above) may be used in a similar manner. However, the use of hafnia nanostraws of comparable size to alumina straws had a higher TE. This may be because the isoelectric point of hafnia is approximately 7, so that in aqueous solution the surface chemistry may be predominantly neutral during delivery. A neutral surface chemistry would not attract charged molecules in solution; hence, the likelihood of clogging cargo would be low.

Hafnia may be reactive ion etched via fluorine-based etched chemistry (CF4) in addition to chlorine-based etch chemistry (BCl3) as with the case of alumina (discussed above). The advantage with fluorine chemistry lies in the relative ease of regulation and maintenance compared to the cost, labor, and risk associated with maintaining chlorine-based etch chemistries.

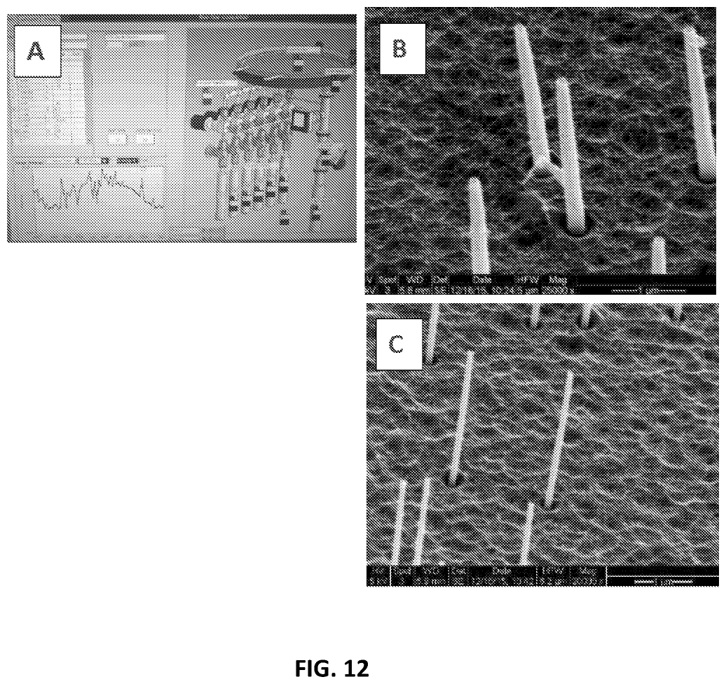

FIG. 12 illustrates hafnia nanostraws. As shown in FIG. 12A, the hafnia nanostraws were successfully fabricated using a Savannah 200 (Cambridge Nanotech) utilizing similar processing parameters as alumina-based nanostraws. In FIG. 12B, hafnia nanostraws exhibit clean, open pores and appear to have uniform diameters along the entirety of the straw length, also shown in FIG. 12C. Hafnia offers the advantages of low-temperature deposition, a relatively neutral isoelectric point, and potential compatibility with fluorine-based etch chemistries.

Nanostraw production may be scaled to higher throughputs, despite the relatively slow ALD rates. The deposition rate in thermally enhanced ALD reactors can be significantly increased by moving from the time-limiting domain to the spatial domain for the dosing of the various process gases in parallel as well as over large substrate areas. Throughputs as high as 3600 ALD coated wafers/hour have been reported. In addition, the use of flexible polymer substrates may allow ALD coating into roll-to-roll processing.

Any of the nanostraws described herein may be formed using an anisotropic dry etching process to etch the top metal oxide layer when forming the nanostraws. For example, dry etching techniques can generate anisotropic etch profiles and have come into favor in recent years for reasons of selectivity and directionality. Etching processes have been generally can be grouped classified into five categories: sputter etching, chemical etching or gasificaiton, accelerated ion-assisted etching, sidewall-protected ion-enhanced etching, and reactive-ion etching.

The term reactive-ion etching has often been used to refer to anisotropic etching; however, this is not entirely correct. In low-density plasmas, i.e. with current densities 0.01 to 1 mA/cm2, there are too few impinging ions to achieve practical etch rates. However, in the more recent high-density plasma-etching systems bombarding ion fluxes, with current densities of 1 to 10 mA/cm2, a sufficient concentration of “hungry ions” may be created to devour substrates. It is for such cases that the term reaction-ion etching is appropriate.

One of the important considerations in plasma etching is the temperature rise in the film/substrate. Plasma-etching species and sputtered atoms that imping on surfaces are far more energetic, for example, than comparable atoms emanating from evaporation sources. During ionic impact, condensation, and reaction, the excess energy liberated must be dissipated via the substrate (as heat dissipation in vacuum is radiative) or otherwise it may heat excessively to the detriment of film quality.

The etching or removal of atoms from film or substrate surfaces that are immersed in plasmas occurs by both physical and chemical means. Changing the ion energies and pressures shifts the dominant material-removal processes. For example, physical sputter etching occurs at the lowest pressures (˜1 mtorr) and highest energies (keV). Ion-assisted etching via the surface damage mechanism takes place at lower energies and somewhat higher pressures (˜50 mtorr). In both cases surface etching tends to be anisotropic. However, with chemical etching at elevated pressures of ˜1 torr, energetic ion bombardment is precluded and the result is isotropic attack of films. Because the mass of many of the ionic species in practical plasma etching processes is large, their motion may not be in phase with the RF field. As a result, the ionic-displacement amplitude and energy are generally too low to cause sputtering.

In the PlasmaQuest ECR etcher, a plasma is created by the electron-cyclotron resonance effect. The microwaves are tuned to the cyclotron resonance frequency of electrons in the gas. They excite the atomic electrons to the point where they gain enough kinetic energy to be stripped off of the atoms, ionizing the gas. This allows plasma to be created without an electrical discharge and without increasing the temperature of the ions in the plasma by a significant degree. The resulting plasma can thus have a low temperature and a low density, and also has a high ionic fraction, which may be useful for plasma etching on polymer substrates, including plasma etching to form the nanostraws described herein.

Fabrication of the nanostraws described herein typically includes oxidation. Electrons, produced by ionization of gas (e.g., 10% O2, 90% Ar), gain energy in the electric field. Subsequent collisions between these energetic electrons and neutral gas molecules result in an energy transfer to the molecules producing chemically active atoms, free radicals, ions and free electrons. The combustion products, which are dissociated and harmless, are carried away in the gas stream outlet. This process occurs near ambient temperatures without employing toxic chemicals and it is highly selective to polymers over metal oxide structures such as alumina, silica, and hafnia.

Provided the mechanism of plasma oxidation, characteristics, i.e. etch rate, selectivity, roughness, etc., may be a function of plasma characteristics such as RF power (W), chamber pressure (mTorr), gaseous partial pressures (mTorr), chamber temperature, and substrate temperature.

Standard etch protocols include four sequential etch cycles which may yield 1.0 to 1.5 nanostraw length (factoring in 30 to 45 degree viewing angle). No clear differential trend in performance has been internally observed, to date, across this nanostraw length range.

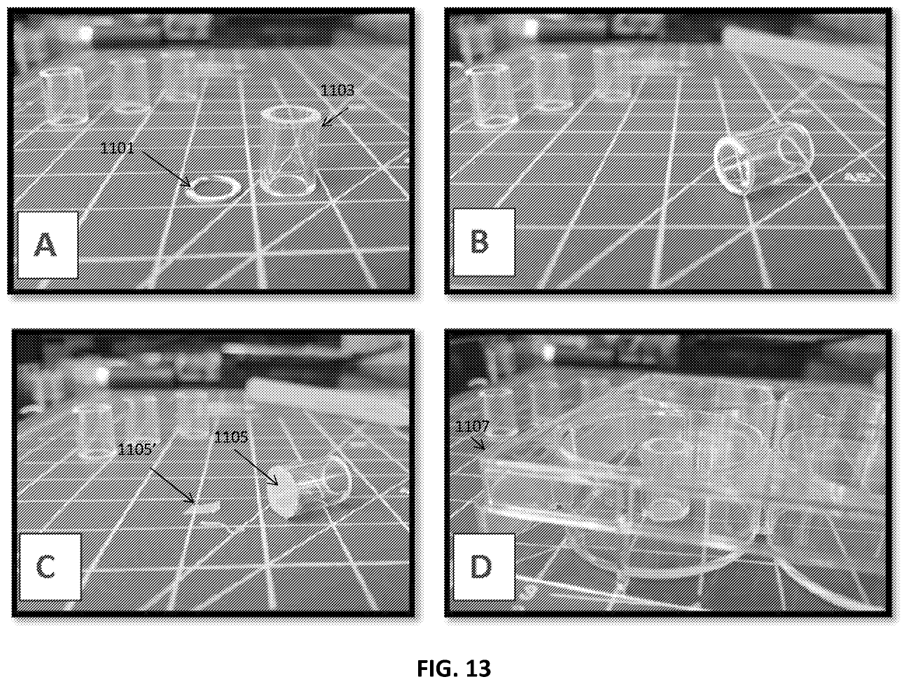

Nanostraw well inserts may be fabricated or assembled using any of the nanostraws described herein. For example, FIGS. 13A-13D illustrate one example of nanostraw assembled using the improved methods described herein. In FIG. 13A, components for a nanostraw well insert apparatus are show, including a double-sided annular tape 1101, a polycarbonate (PC) tube 1103 and the nanostraw membrane 1105. Device assembly entails cutting or forming the PC tube, and cutting or forming the double sided tape to the respective dimensions. In FIG. 13B, the double-sided tape may be mated with the tube, and then sticking the tube to the nanostraw membrane (oriented with the nanostraws facing into the tube). Optionally, as shown in FIG. 13C, the excess material 1105′ may be trimmed. FIG. 13D illustrates a nanostraws well insert devices integrated into an existing culture well plate 1107. Thus, the assembly of nanostraw well inserts may include: taping one side of the double-sided annular tape to one end of a polycarbonate tube-well; taping the opposite side of the double-sided tape to the nanostraw membrane (nanostraws normal to device); and optionally cutting and trimming excess nanostraw membrane from edges of nanostraw well insert using standard trimming tools.

Following assembly, the nanostraw well insert devices may be cleaned with 70% ethanol in water, air dried for 10 to 15 minutes, and sterilized in UV light for 15 minutes. Nanostraw well inserts may fit readily into existing 6-, 12- and 24-well plates, and are imaging friendly.

The cutting/trimming process may be batch performed at-scale with metal die cutters, or with CO2 laser welding directly to polycarbonate tube-wells. Further, the dimensions of the tube wells may likely be designed taking into account ergonomic considerations.

Any of the apparatuses, including any of the nanostraw well insert devices described herein may be used for cell culture, and particularly long-term cell culture (e.g., longer than five days), and used at any time to access the internal structure of the cell (e.g., to transfect material into the cell. Unlike previously described and characterized nanostraw devices, these apparatuses may be used for longer than 5 days, including up to at least 3 weeks, without significantly effecting cell viability; further, at any time during this period the nanostraw structures may be used to access (e.g., deliver material, remove material, etc.) internal cell materials.

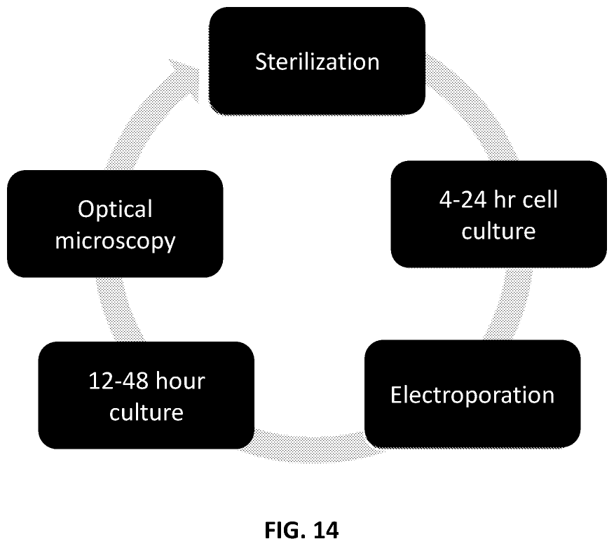

For example, FIG. 14 illustrates a short-term prior standard cell culturing protocol for a model cell line (e.g., such as CHO, HEK293T, HeLa, etc.). After assembly, a nanostraw well insert device may be sterilized, e.g., with 70% ethanol in water, air dried for 10 to 15 min and placed under UV light (λ=305 nm) for 15 min. Generally speaking, a standard protocol may entail culturing cells on top of the nanostraw membrane for more than 4 hours (typically, an over-night culture) in 350 μl of “regular” media (e.g., 10% Fetal bovine serum (FBS), DMEM media from Invitrogen company (Cat #10564) plus 1× Penicillin/Streptomycin (P/S)). A nanostraw well insert apparatus may be placed in a 24-well plate containing a bath of 350 μl of 1× regular media (dispensed external to the nanostraw device). After electroporation/transfection, cells are cultured for an additional 12-48 hours prior to analysis via optical microscopy.

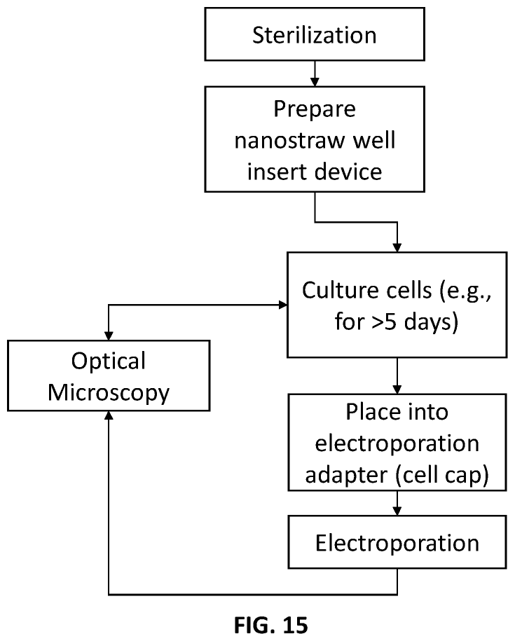

Because the apparatuses, including formed as described herein have been shown to have particularly long viability time periods (e.g., exceeding five days) for tissue culture, particularly as compared to those previously described, including in particular alumina nanostraws, cells may be cultured for an extended period of time. For example, FIG. 15 illustrates one method for using the nanostraw well insert apparatuses described herein. In FIG. 15, the insert is first sterilized, and may then be prepared for cell culture. For example, the cell culture medium may be added to the inside of the insert deice, and the insert may be placed into a tradition multi-well dish, for which it is well adapted. Additional cell culture medium (the same or a different medium) may be added to the dish into which the insert is placed, and a cover provided. Cells may also be added into the insert device. Thereafter, the cells may be incubated for a desired period of time, including in particular very long times (>5 days). This is in contrast to prior variations of the nanostraw devices, for which cell viability dropped dramatically after 3-4 days (e.g., with less than half of the cells surviving beyond this period. The apparatuses described herein may have >50% (e.g., >60%, >70%, etc.) viability after 5 days.

In addition to being well adapted for use with a multi-well dish, as discussed above, the nanostraw well insert devices described herein may be adapted specifically for use with an electroporation adapter that may securely hold the nanostraw well insert device. Electroporation adapters (also referred to herein as “cell caps”) are described in greater detail below, and may generally have a cylindrical body into which the insert device may be inserted and held above a cargo region calibrated so that the bottom of the nanostraw well insert device does is separated from a base electrode by a predetermined distance that is also configured to prevent bubbles/vapor formation between the base electrode and the bottom of the insert device. A cap portion may then be placed over the cylindrical body so that a second (top) electrode is held a fixed, predetermined distance from the bottom electrode, and projects into the insert device. The walls of the electroporation adapter (cell cap) may be insulated (e.g., thermally insulated, electrical insulated, etc.). The cap may also seal the apparatus so that it may be transported (e.g., to an electroporation apparatus), and may include external contacts in communication with the top electrode and base electrode, so that electroporation may be performed from outside of the apparatus, controllably transporting a cargo (e.g. plasmid, protein, etc.) from the cargo solution region above the base electrode, though the nanostraws, and into the cells.

To optimize the nanostraw well insert devices and the electroporation adapters (cell cap), factors believed to affect transfection efficiency (TE) and cell viability (CV) were quantified. Among these tested factors were: confluency, top electrode geometry, electrode-electrode distance, applied voltage and stimulus duration, osmolarity in the top solution, and washing media before electroporation. Confluency was tested from 25,000 cells to 200,000 cells with the best TE and CV with 25,000 cells.

The electrode distance was observed to play a critical role with respect to the field strength delivered to cells on Nanostraw devices. Distances of 0.25 cm to 1.25 cm were examined with 0.5 cm showing relatively higher TE and CV. TE and CV were inversely related with increasing field strengths (i.e., shorter electrode-electrode distance, higher applied voltage, and longer stimulation duration). Model cells lines survived up to 15V of applied voltage with greater than 80% CV; however, cell death increased rapidly above this threshold voltage. Electrode geometry was not observed to have a significant contribution to performance amongst planar, point, and spiral designs. Exchanging for fresh media directly before electroporation with a solution of lower osmolarity (PBS vs “regular” media) indicated healthier cells and higher transfection rates.

Thus, in some variations, the use of electrodes built into the nanostraw well insert devices and/o the electroporation adapters may be important in enhancing both TE and CV. For example, separation between the top electrode and the base electrode (with the nanostraw substrate between the two, in some cases may optimally be between 0.25 cm to 1.25 cm, e.g., between 0.3 cm and 0.8 cm. Outside of this range (e.g., >1.25 cm), cell viability, particularly for longer culture times, fell off sharply.

As an example, an electroporation transfection protocol using nucleic acids in model cell lines was used to examine the apparatuses described herein. Prior to each transfection via electroporation, the bottom Pt electrode (base electrode) was cleaned with 70% ethanol to water using a standard Kimwipe. 60 μl of liquid delivery precursor (e.g., the cargo solution, such as nucleic acid, plasmid, protein, fluorescent dye, Co+2, etc.) was dropped into the delivery reservoir (see FIG. 2A). The pre-cultured nanostraw well device may be placed into a delivery precursor holder (e.g., an electroporation adapter/cell cap) and slight pressure may be applied to ensure complete wetting of the liquid delivery precursor to the bottom of the nanostraw membrane. The top Pt electrode was then lowered into the nanostraw well device solution at approximately 0.7 cm above the bottom Pt electrode. From past observation, it is important to allow 1 minute prior to applying electricity. This is to ensure complete wetting of the nanostraws membrane with delivery precursor via simple diffusion.

After allowing time for diffusion, DC electricity was applied across the nanostraw well device (e.g., across the electroporation holder/cell cap). The standard electrode configuration is negative and positive for the bottom and top electrodes, respectively. Negatively charged precursor may be electrically driven upwards into the top solution and cell culture. The standard DC pulse profile is as follows: voltage of 10 V, individual pulse width of 200 μs, frequency of 20 Hz, total time duration of 40 seconds. After pulsing electricity, the nanostraw well device should remain in contact with the delivery precursor for an additional minute to allow for diffusion of species into opened electropores. The device is then placed back in the incubator for an additional 12 to 48 hours prior to imaging.

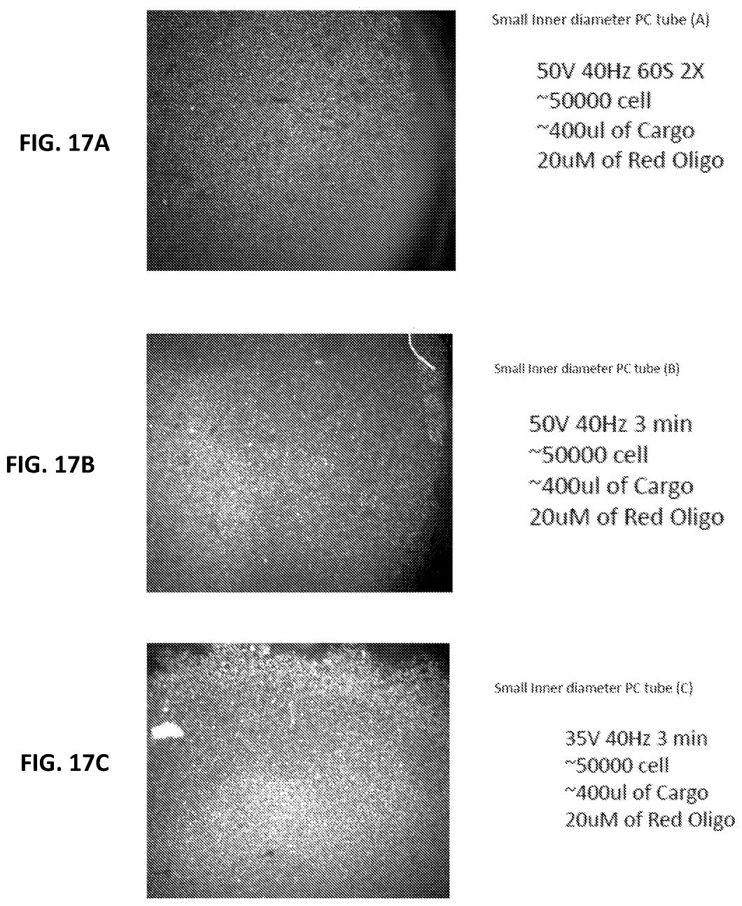

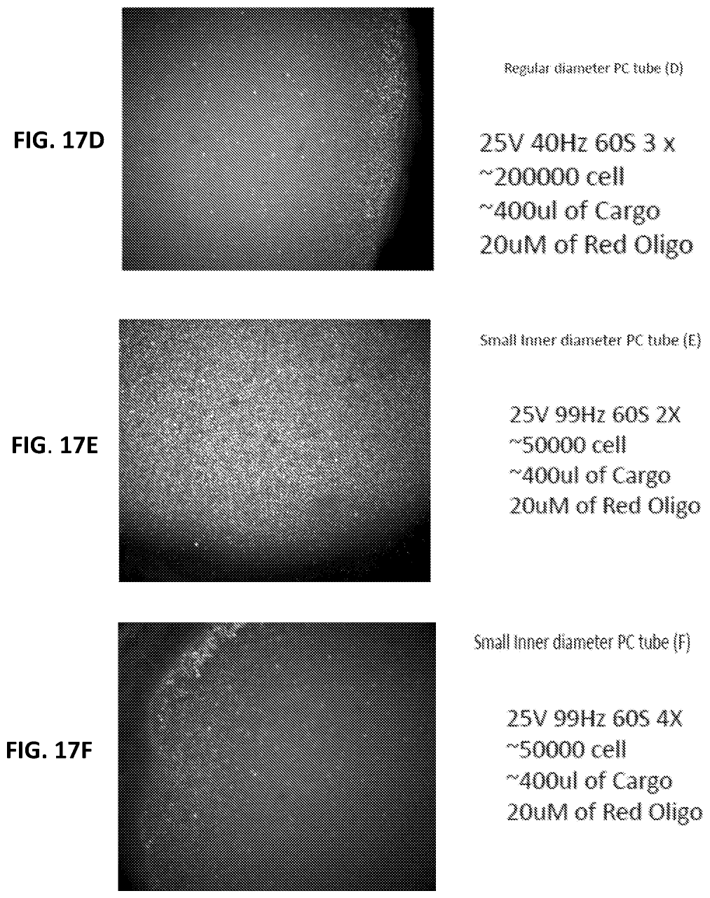

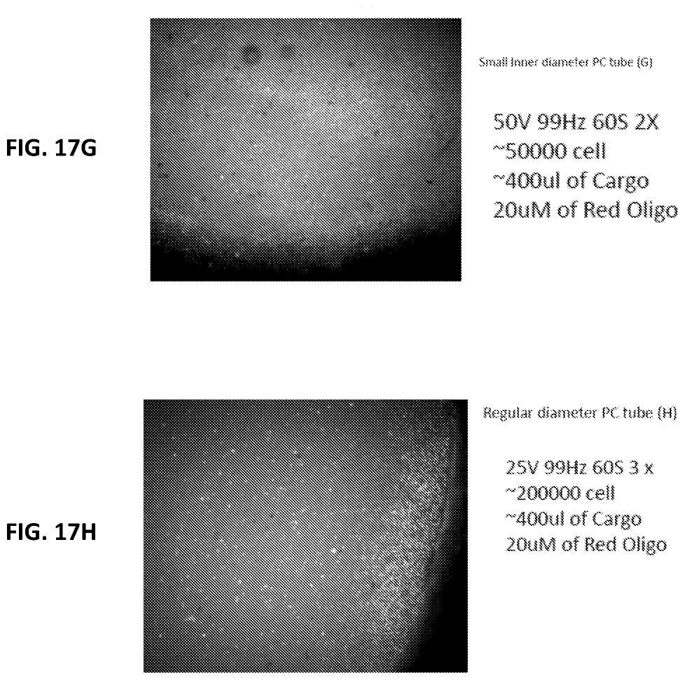

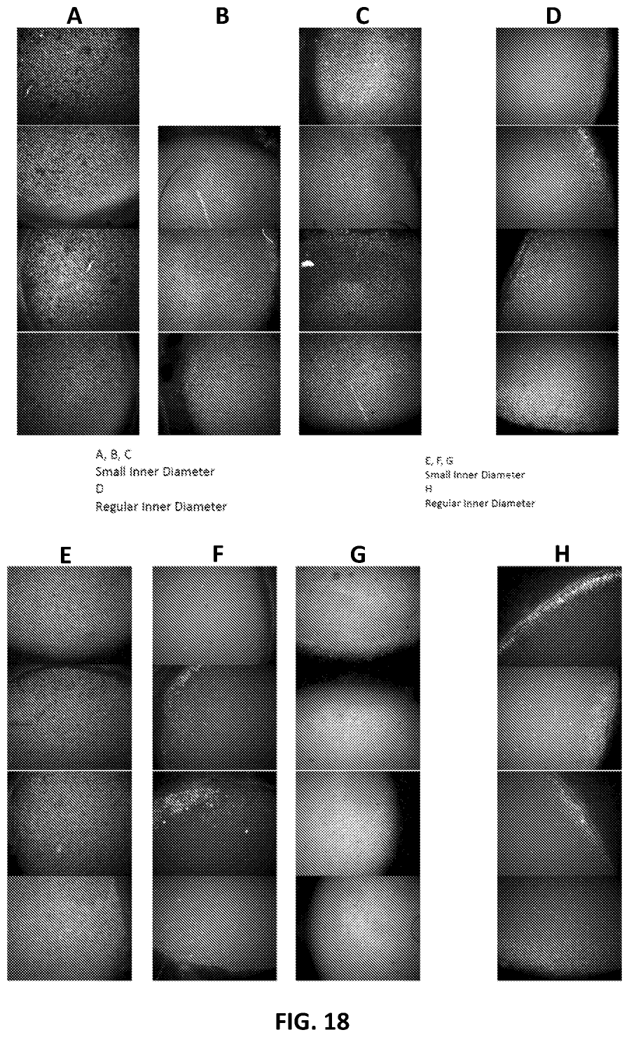

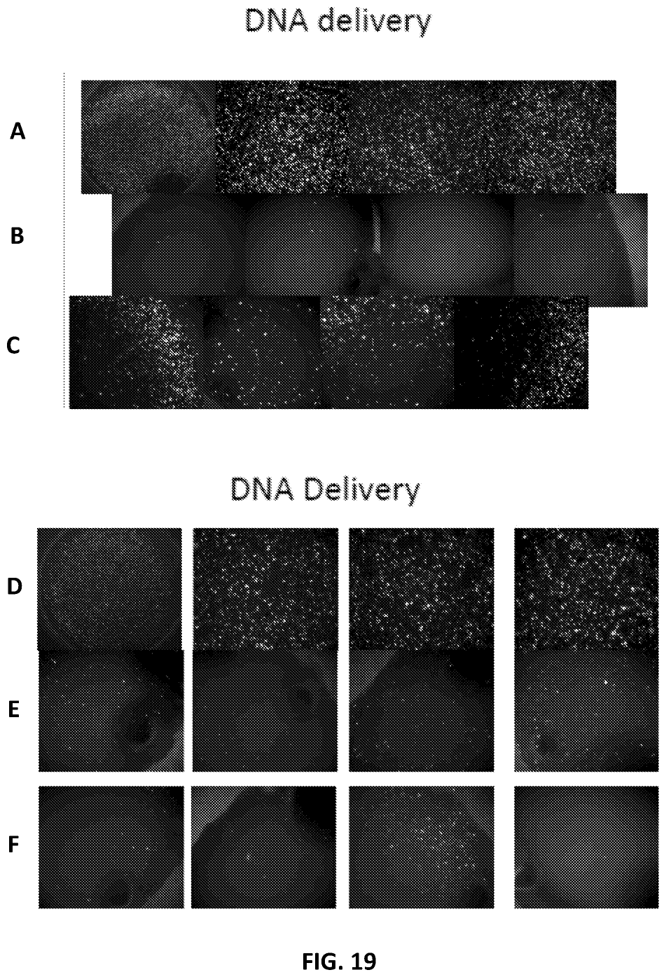

FIG. 17A-FIG. 17H, FIG. 18A-FIG. 18H, and FIG. 19A-FIG. 19F illustrate delivery of nucleic acids into model cell lines can be enhanced via fabrication and experimentation parameter optimization as discussed above.

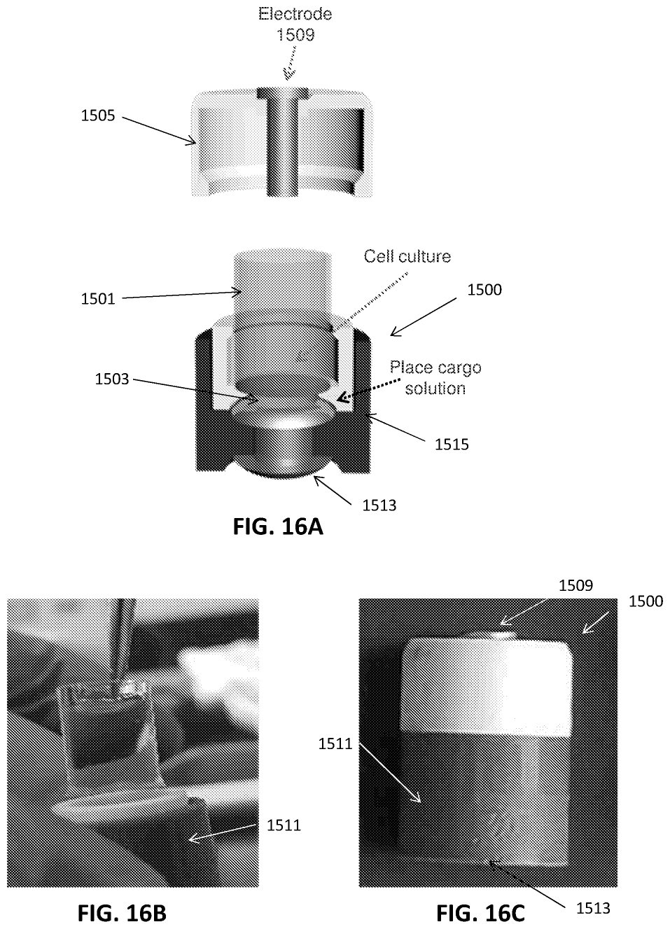

Turning now to FIG. 16A-16C, an apparatus including a nanostraw well insert device and a compatible/matching delivery precursor holder (e.g., an electroporation adapter/cell cap) are shown. FIG. 16A shows a nanostraw well insert 1501 inserted into a holder (also referred to as an electroporation adapter or cell cap 1500. The nanostraw well insert is substantially as described above in FIGS. 1A-1B and 2A. The electroporation adapter is configured to secure the insert within an elongate cylindrical body 1515, so that the bottom of the insert is flat and parallel with the base electrode 1513. The base electrode is secured across the bottom of the adapter and may be flat or concave, to hold and form the reservoir depot 1503. As mentioned, the adapter may be configured to prevent bubbles from getting trapped between the membrane of the nanostraw well insert and the base electrode; for example, the adapter may include one or more channels in the side and/or through the side to allow air to escape from between the flat bottom of the membrane in the insert and the depot 1503.

In general, the electroporation adapter is configured to hold the insert securely, within the cylindrical chamber, and a cap 1505 including a top electrode 1509 may be attached over the insert when it is held within the device. The cap may be held on and in place by a friction fit and/or a mechanical, magnetic, or other attachment (e.g., screwed on, snapped on, etc.). The cap my in particular, hold the top electrode in position over and at least partially into the insert. The entire electroporation adapter may maintain the separation between the top electrode and the base electrode as optimally described here (e.g. between 0.3 and 0.8 cm, or about 0.5 cm).

The top electrode 1509 and base electrode 1513 may be positioned within the adapter as shown, but may include one or more connections to electrical contacts on the outside of the adapter so that even as the insert is held within the sterile internal chamber of the adapter, the entire apparatus may be manually held and manipulated, including placing into an electroporation apparatus for applying current across the electrodes as described above.

In FIGS. 17A-17H, the electro-delivery results of cargo composed of 20 uM red oligo in 400 ul TE buffer is shown for different configurations of wells and/or different electrical parameters. In general, the electro-delivery of cargo into suspended inside the wells (using a stylus electrode on top and a band electrode, e.g., a ring electrode, at the base as described herein resulted in significant take-up and expression of the cargo, with little toxicity. FIG. 17D and FIG. 17H) show electro-delivery results using regular-sized wells and a slab electrode on top and a ring electrode at the base. FIG. 17A shows the results of the application of 50V, at 40 Hz for 60 seconds (repeated twice) applied to approximately 50,000 cells. Similarly, FIGS. 17B and 17C show the application of 50V and 35V, respectively, for 3 minutes to approximately 50,000 cells. FIG. 17D shows 25V applied at 40 Hz for 60 seconds (three times), in 200,000 cells while FIGS. 17E and 17F show the results of 25V applied at 99 Hz for 60 seconds, two times or four times, respectively, in approximately 50,000 cells. FIG. 17G shows the results of applying 50V at 99 Hz for 60 seconds, twice, in approximately 50,000 cells. Finally, FIG. 17H shows the results of applying 25V at 99 Hz for sixty seconds, three times, in 200,000 cells.

FIG. 18 show multiple images of cells into which a cargo of 20 uM red oligo in 400 ul TE buffer is delivered into cells as described herein, using different parameters, including different wells (e.g., petite wells, regular wells, etc.) using a stylus electrode on top and a ring electrode at the base as described herein. In the columns corresponding to FIGS. 18A, 18B, 18C, 18E, 18F and 18G wells having a small inner diameter were used for electro-delivery with a stylus electrode on top and a ring electrode at the base at various voltage and frequencies. FIGS. 18D and 18H show regular inner diameter wells used for electro-delivery with a slab electrode on top and a ring electrode at the base. As described above, cargo was more successfully delivered in some configurations as compared with others.

Similarly, FIGS. 19A-19F show the electro-delivery expression results of cargo composed of 10 ug of pMaxEGFP plasmid resuspend in 30 ul of buffer TE (FIGS. 19A, D) using regular cell pac for control or 50 ug of pMaxEGFP plasmid resuspend in 400 ul of buffer TE (FIGS. 19B, 19C, 19E, and 19F) into cells using a ring electrode.

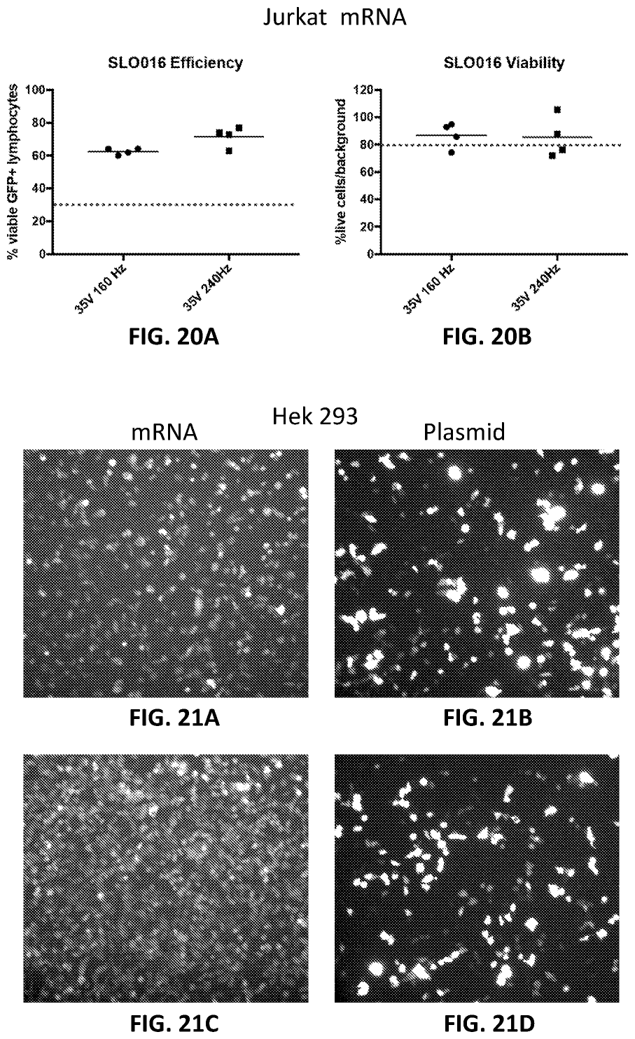

FIGS. 20A and 20B summarize the effects of various electro-delivery of cargo (e.g., mRNA of green florescent protein, GFP) in Jurkat cells, using either 35V at 160 Hz or 35V at 240 Hz. In both sets of conditions the percent efficiency was greater than 60% and was greater than 70% at 240 Hz. (FIG. 20A). Further, the electro-delivery of cargo using the nanostraw well devices described herein did not substantially reduce the viability of the cells that were treated, as shown in FIG. 20B. In this example, the viability was greater than 75% (and on average, greater than 80%) regardless of the parameters used for electro-delivery of the cargo into the cells.

FIGS. 21A-21D are images showing the high level of cells taking up the cargo, either mRNA (FIGS. 21A and 21C) or plasmid (FIG. 21B or 21D) when Hek 293 cells were treated as described herein, using a small-well nanostraw well device.

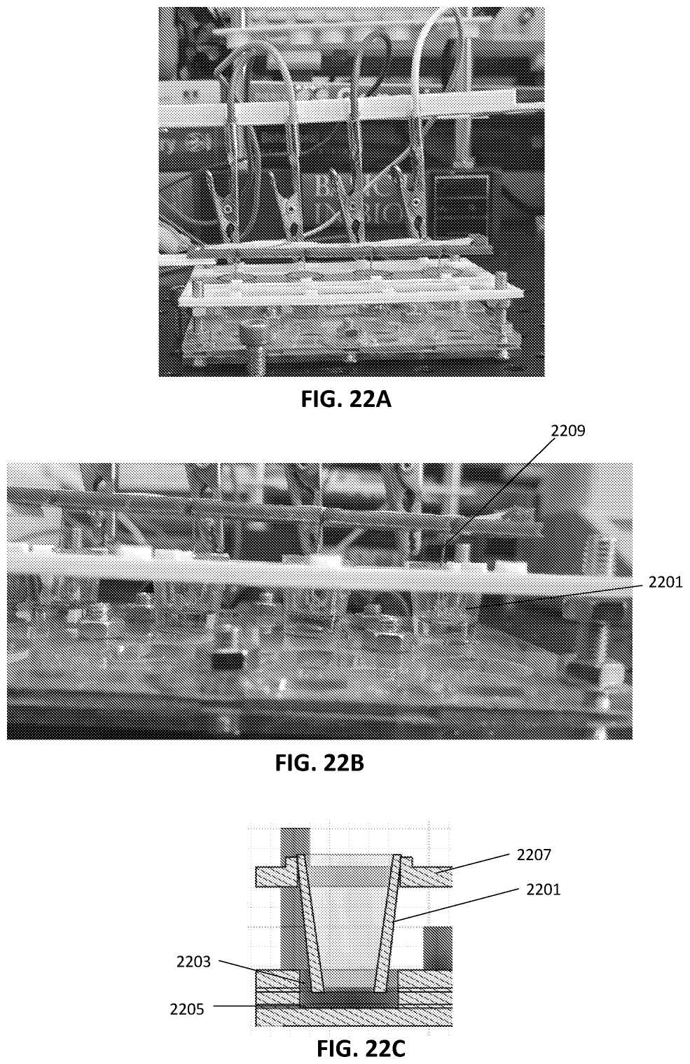

FIGS. 22A-22C illustrate one example of a system for electro-delivery of a cargo (e.g., a polynucleotide) as described. In FIG. 22A, the system includes multiple (e.g., four are shown) parallel nanostraw well devices that may be used to culture and/or transfer cargo into the cells as described herein. In FIG. 22A, the four nanostraw well devices 2201 all have tapered wells and are placed into fluid reservoir depots 2203. The base of the reservoir depot is a plate electrode formed into a ring 2205. Stylus electrodes 2209 are inserted into the top of the wells, which are supported by a holder 2207.

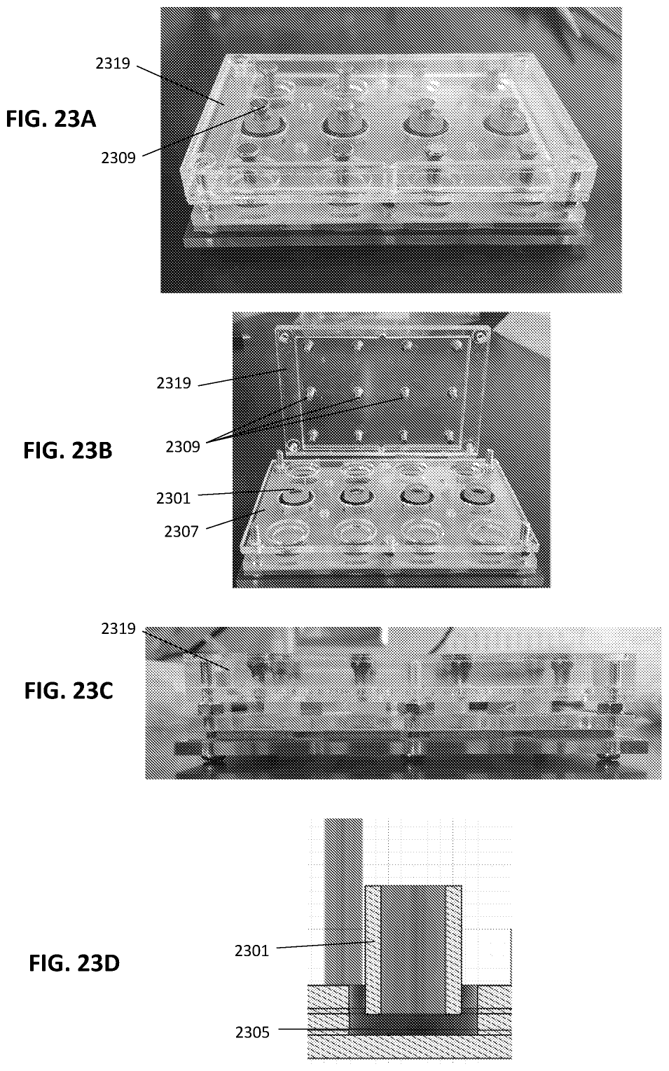

FIGS. 23A-23D show an alternative system configuration in which cells may be cultured and treated as described herein. The plurality (four are shown) of nanostraw well devices 2301 are each held within a fluid reservoir depot formed in the base. The base also includes an array of band electrodes 2305 cut from a metal plate. A mount 2307 may hold the nanostraw well devices in the fluid reservoirs, and a cover 2319 may be placed over the top of the system. The cover may include one or more openings therethrough, including an opening to allow electrodes (e.g., rod electrodes 2309), as shown, access into the nanostraw well devices. The systems shown in FIGS. 22A-22C and 23A-23D may be used to treat a large number of cells.

Any of the methods (including user interfaces) described herein may be implemented as software, hardware or firmware, and may be described as a non-transitory computer-readable storage medium storing a set of instructions capable of being executed by a processor (e.g., computer, tablet, smartphone, etc.), that when executed by the processor causes the processor to control perform any of the steps, including but not limited to: displaying, communicating with the user, analyzing, modifying parameters (including timing, frequency, intensity, etc.), determining, alerting, or the like.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

In general, any of the apparatuses and methods described herein should be understood to be inclusive, but all or a sub-set of the components and/or steps may alternatively be exclusive, and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

1-41. (canceled)

42. A nanostraw cell culture system, the system comprising:
an adapter configured to hold a nanostraw well insert device, the nanostraw well insert device comprising a membrane from which a plurality of nanostraws project by at least 0.1 microns, the adapter comprising:
a chamber having a base;
a band electrode on in the base,
wherein the chamber is configured to securely hold the nanostraw well insert device so that the membrane is vertically offset from the base so that the plurality of nanostraws is in fluid communication with a reservoir depot between the base and the membrane, further so that the band electrode is laterally offset from the plurality of nanostraws;
a cover comprising a top electrode;
a first electrical contact on or in the adapter in electrical communication with the band electrode; and
a second electrical contact on or in the adapter in electrical communication with the top electrode.

43. The system of claim 42, wherein the band electrode comprises a ring electrode.

44. The system of claim 42, wherein the band electrode is laterally offset from the plurality of nanostraws on the membrane.

45. The system of claim 42, wherein the adapter is configured so that the band electrode does not underlie the plurality of nanostraws when the nanostraw well insert device is held within the adapter.

46. The system of claim 42, further comprising a nanostraw well insert device comprising a membrane from which a plurality of nanostraws project by at least 0.1 microns.

47. The system of claim 42 wherein the chamber is configured to securely hold the nanostraw well insert device so that an inner diameter of the band electrode is larger than an outer diameter of the membrane.

48. The system of claim 42 wherein an outside diameter of the band electrode is smaller than an inside diameter of the base.

49. The system of claim 42 wherein an inside diameter of the band electrode is greater than 3 mm.

50. The system of claim 42 wherein the band electrode is laterally offset from a perimeter of the membrane by at least 2 mm when the nanostraw well insert device is held within the adapter.

51. The system of claim 42 wherein the band electrode is substantially round.

52. The system of claim 42, wherein the top electrode comprises a stylus electrode.

53. The system of claim 42, wherein the top electrode has a diameter of less than 2 mm at its widest dimension horizontal dimension.

54. The system of claim 42, wherein the top electrode has a surface area of less than about 4 mm² at its widest dimension horizontal dimension.

55. The system of claim 42, wherein the top electrode has a surface area of less than about 2 mm² at its widest dimension horizontal dimension.

56. The system of claim 42, wherein the top electrode is separated from an inner surface of the adapter by at least 1 mm.

57. The system of claim 42, wherein the top electrode comprises a slab electrode.

58. The system of claim 42, wherein the base is configured to vent air between the reservoir depot a bottom of the nanostraw well insert.

59. The system of claim 42, wherein the adapter further comprises an inlet configured to regulate a pressure of the reservoir depot in the base.

60. A method of delivering a material into one or more cells, the method comprising:
culturing one or more cells on a nanostraw well insert device comprising a well, wherein the nanostraw well insert device comprises a membrane, wherein a plurality of nanostraws project through the membrane and into the well by greater than 0.1 microns;
placing the nanostraw well insert device into a chamber of an adapter so that the membrane is vertically offset from a base of the chamber with the plurality of nanostraws in fluid communication with a reservoir depot at the base, wherein the reservoir depot is in electrical communication with a band electrode so that the band electrode is laterally offset from the membrane;
placing a cover over the chamber, so that a top electrode on or in the cover is within the chamber and is separated from the band electrode with the nanostraw well insert therebetween;
applying electrical energy between the band electrode and the top electrode to deliver a material from the reservoir depot, through the plurality of nanostraws and into the one or more cells; and
removing the nanostraw well insert from the adapter and culturing the one or more cells.

61. The method of claim 60, wherein applying the electrical energy comprises applying a voltage between about 20V and 110 V at a pulse width of between 10 and 500 microseconds at a pulse frequency of between 30 Hz and 110 Hz.
